(19) United States
(12) Patent Application Publication
(10) Pub. No.: US 2023/0036213 A1
(43) Pub. Date: Feb. 2, 2023

(54) T-CELL EPITOPES OF HUMAN PARAINFLUENZA VIRUS 3 FOR ADOPTIVE T-CELL IMMUNOTHERAPY

(71) Applicant: CHILDREN'S NATIONAL MEDICAL CENTER, Washington, DC (US)

(72) Inventors: Katherine HARRIS, Washington, DC (US); Michael KELLER, Washington, DC (US); Catherine BOLLARD, Washington, DC (US)

(73) Assignee: CHILDREN'S NATIONAL MEDICAL CENTER, Washington, DC (US)

(21) Appl. No.: 17/784,072

(22) PCT Filed: Dec. 10, 2020
(86) PCT No.: PCT/US2020/064286

§ 371 Date: Jun. 9, 2022

Related U.S. Application Data
(60) Provisional application No. 62/946,416, filed on Dec. 10, 2019.

(51) Int. Cl. A61K 35/17; A61K 39/12; C07K 14/115; C12N 5/0783; A61K 39/39

(52) U.S. Cl. CPC A61K 35/17; A61K 39/12; C07K 14/115; C12N 5/0636; A61K 39/39; A61K 2039/5158

(57) ABSTRACT

Disclosed are compositions of T-cells, libraries of such T-cells, and methods of making T-cell subpopulations for treatment of human parainfluenza virus (HPIV) infections, particularly HPIV type 3 (HPIV3) infections. Also disclosed are T-cell compositions comprising cell subpopulations stimulated with HPIV3 antigens and T cell banks containing these compositions for off-the-shelf availability of T cells for treatment of disease.

### CROSS-REFERENCE TO RELATED APPLICATION

This application claims priority to U.S. Provisional 62/946,416, filed Dec. 10, 2019 which is incorporated by reference in its entirety.

### GOVERNMENT SUPPORT

This disclosure was made with funding from the US government under US Grant No. K23-HL136783-01 provided from the National Institutes of Health under the Department of Health and Human Services. The United States government may have rights in this disclosure.

### FIELD OF THE INVENTION

The present disclosure pertains to the fields of infectious disease, virology, and cellular immunology. It provides non-engineered adoptive T-cell compositions, therapies, and processes of manufacture that are tailored for treatment or prevention of a subject with a human parainfluenza virus type 3 (HPIV3) infection and in some embodiments, for those subjects who are immunocompromised, and subjects who have, are diagnosed with, or suspected of having primary immunodeficiency disorders. The present disclosure also extends to methods of manufacturing such non-engineered adoptive T-cell compositions and to the generation of single antigen targeting T-cell banks from healthy donors that provide for personalized T-cell therapy. Also provided are uses of such non-engineered adoptive T-cell compositions for treatment of HPIV1 infections.

### BACKGROUND

Human parainfluenza viruses (HPIVs) are enveloped, single-stranded negative-sense RNA viruses in the Paramyxoviridae family classified into 4 major subtypes (HPIV 1-4) which cause acute respiratory infections (ARIs) in all ages, but notably in infants and young children.

HPIVs remain the second main cause of hospitalization in children under 5 years of age who are suffering from respiratory illnesses with HPIV3 infections accounting for the majority of these hospitalizations. HPIV 1 and 2 are more commonly associated with upper respiratory illnesses (URIs) such as croup while HPIV3 usually causes lower respiratory illnesses (LRIs) including pneumonia and bronchiolitis. Despite the prevalence of HPIV infection at a young age, reinfection is common. In developing regions of the world, infants and young children are at the highest risk of mortality either from primary HPIV3 viral infection or from secondary consequences such as bacterial infections.

HPIV infections in healthy children most commonly cause self-limited, mild ARIs. However, children with immunodeficiencies due to genetic disorders, solid organ transplants or hematopoietic stem cell transplants (HSCT) are at increased risk for severe HPIV infections or other viral infections.

Such viral infections remain a major cause of morbidity and mortality in children and in adult patients following hematopoietic stem cell transplantation (HSCT). In immunocompromised patients, respiratory viruses, including human parainfluenza virus, cause significant pulmonary disease and pneumonias. HPIV3 is the most common serotype isolated from immunocompromised patients having lower respiratory tract infections. HPIV3 infection can be fatal and in some instances is associated with neurologic diseases such as febrile seizures. It can also result in airway remodeling which is a significant cause of morbidity.

Given the considerable morbidity and mortality associated with HPIV3 in immunocompromised patients, there is a significant need for developing preventative and treatment strategies for treatment of this virus. Nevertheless, there is currently no approved vaccines or antiviral therapies for prevention or treatment of HPIV3 infections.

Following HSCT, there is a significant delay in the recovery of T-cell immunity which is highly associated with viral reactivation and disease relapse. Accordingly, the development of adoptive cellular immunotherapy to restore antiviral immunity was an attractive treatment objective for the inventors. Adoptive transfer of virus-specific T-cells (VST) has proven to be a safe and effective strategy for prevention and treatment of other some viral infections after HSCT such as those associated with Epstein Barr virus (EBV), cytomegalovirus (CMV), adenovirus (ADV), BK virus (BKV), and Human herpesvirus-6B (HHV6B). However, to date, adoptive T-cell therapy has never been attempted or been found effective for prevention or treatment of HPIV3 infection.

The inventors believed that the knowledge and characterization of HPIV viral antigens recognizable by T cells was necessary to enable production of VST targeting HPIV3 and other HPIV infections. The HPIV3 genome encodes only 6 known viral products: two surface glycoproteins (hemagglutinin-ceramidase (HN) glycoprotein and the fusion protein (Fuss); a structural Matrix (Mat) protein, which is critical for vision assembly and budding; a nucleocapsid protein (NP); and the P phosphoprotein and L protein, which interact to form the viral RNA polymerase. Generation of HPIV3-specific T-cells from healthy donors has been recently demonstrated by priming peripheral blood mononuclear cells (PBMCs) with overlapping pools of 15-mer peptides encompassing the HPIV3 HN, Fus, Mat and NP proteins: McLaughlin et al., Cytotherapy, 2016 December; 18(12):1515-1524 (incorporated by reference). However, further identification and characterization of HPIV3 antigens is necessary to attempt to exploit its application to adoptive T cell therapy and to prove its feasibility and efficiency. As disclosed herein, the inventors sought and identified several immunodominant epitopes of HPIV3 antigens useful for producing potent HPIV-specific T-cells for adoptive therapy of HPIV3 infection.

### SUMMARY OF THE INVENTION

The present disclosure relates to adoptive immunotherapy, compositions comprising isolated and primed T-cells to treat an HPIV3 infection, and compositions that comprise HPIV3-specific T-cells primed and expanded ex vivo which enable new therapeutic strategies for prevention and treatment of HPIV3 infection. The disclosure relates to methods of generating HPIV3-specific T-cells from peripheral blood of healthy donors using a good manufacturing practice (GMP)-compliant methodology. The disclosure also relates to methods of stimulating an antigenic-specific cellular immune response and to methods of stimulating an immunodominant antigen-specific immune response against newly identified epitopes within HPIV3 antigens, including, but not limited to, the matrix (Mat) protein antigen. The present disclosure relates to a cell comprising a T-cell receptor specific for HPIV3 antigens, including but not limited to the Mat antigen or functional fragments thereof.

One aspect of the disclosure is directed to a method for preventing or treating an infection with human parainfluenza virus 3 (“HPIV3”), comprising administering to a subject in need thereof an isolated subpopulation of T cells recognizing one or more peptide epitopes of HPIV3. This method may also be directed to treatment of other HPIV infections including HPIV1 especially when cross-reactivity and/or HLA restriction exists with the HPIV3 epitopes disclosed herein.

This method may be used to treat, ameliorate or reduce the severity of at least one symptom of HPIV infection including infection by HPIV1 or 3. Alternatively, the method may be used to prevent infection or spread of infection by HPIV by administering T cells recognizing restricted T cell epitopes or their cross-reactive variants to a subject who is asymptomatic or who is at risk of exposure to HPIV3, HPIV1 or other HPIVs.

In some embodiments of the method as disclosed above, the subpopulation of T cells is in vitro or ex vivo primed to an HPIV3 peptide epitope or cross-reactive HPIV1 or other cross-reactive HPIV epitope and/or expanded prior to said administering.

In some embodiments of this method the T cells are autologous to the subject. In other embodiments, the T cells are allogeneic to the subject and comprise at least one, two, three, four or more HLA class I or class II alleles in common with the subject. Such alleles include those encoded by the HLA-A locus, HLA-B locus, HLA-D locus. HLA-Dr locus, HLA-DQ locus or HLA-DP locus, including HLA-DRA locus and HLA-DRB locus. The HLA-DRB locus encodes HLA-DR1 to HLA-DR17 gene products which may be expressed by a subject or T cell donor.

In the method disclosed above, the T cells can share with the subject at least one class I allele selected from those encoded at the HLA-A, HLA-B or HLA-C loci, or at least one HLA class II allele selected from the group consisting of those encoded at the HLA-DR, HLA-DQ or HLA-DP loci. In some embodiments, the T cells share at least one HLA class II allele selected from the group consisting of HLA-DRB3*02.02, HLA-DRB1*01.01, HLA-DRB1*01.01, HLA-DRB1*01.01, HLA-DRB1*01.01, HLA-DRB1*01.01, HLA-DRB1*01.01, HLA-DRB1*01.01, or HLA-DRB1*01.01.

In some embodiments of this method the one or more peptide epitopes of HPIV3 are contained within (shorter length), or contiguous with (same length), Peptide 84, Peptide 83, Peptide 82, Peptide 59, Peptide 85, Peptide 60, Peptide 76, Peptide 78, Peptide 38, Peptide 77, Peptide 39 or Peptide 50; or wherein the one or more peptide epitopes of HPIV3 comprise one or two insertions, substitutions or deletions to an amino acid sequence of Peptide 84, Peptide 83, Peptide 82, Peptide 59, Peptide 85, Peptide 60, Peptide 76, Peptide 78, Peptide 38, Peptide 77, Peptide 39 or Peptide 50. In other embodiments, the T cell epitopes may form a segment of a longer HLA restricted peptide comprising the sequences of any of the above peptides.

In some embodiments of the above method at least two, three, four, five, or more isolated subpopulations T cells that recognize at least different peptide epitopes of HPIV3 are administered to the subject in need thereof, and wherein at least one of the peptide epitopes is contained in Peptide 84, 83 or 82. Thus, a subject may be administered a combination of T cells each recognizing one of Peptide 84 and 82, Peptide 84 and 83, Peptide 84, 83 and 82, or other combinations of the 12 peptide epitopes disclosed herein.

This method may involve administration of T cells which recognize one or more peptide epitopes of HPIV3 contained within Peptide 84, Peptide 83, Peptide 82, Peptide 59, Peptide 85, Peptide 60, Peptide 76, Peptide 78, Peptide 38, Peptide 77, Peptide 39 or Peptide 50; or which recognize one or more peptide epitopes of HPIV3 comprising one, two, three, four, or five insertions, substitutions or deletions to an amino acid sequence of Peptide 84, Peptide 83, Peptide 82, Peptide 59, Peptide 85, Peptide 60, Peptide 76, Peptide 78, Peptide 38, Peptide 77, Peptide 39 or Peptide 50. In some embodiments the T cells are optionally contacted with said one or more peptide epitopes in vitro or ex vivo, and optionally expanded, prior to said administering.

In some embodiments, the T cells comprise one or a plurality of banked T-cell subpopulations that recognize HPIV3, HPIV1 and/or other HLA-restricted HPIV peptide epitopes. In other embodiments, T cells that recognize epitopes or other respiratory pathogens may be incorporated with the subpopulations of T cells recognize HLA-restricted HPIV epitopes.

Other embodiments of this method may further comprise determining HLA subtype(s) of the subject and/or donor (used to produce the HPIV specific T cells) and selecting one or a plurality of banked T-cell subpopulations having activity against an HPIV3, HPIV1 or other HPIV peptide epitope restricted by said HLA subtype(s), and administering said selected T cell subpopulations to the subject.

Another aspect of this disclosure is directed to at least one isolated subpopulation of T cells recognizing one or more restricted peptide epitopes of HPIV3, HPIV1 or other HPIV. Such a composition may comprise at least one isolated subpopulation of T cells was in vitro or ex vivo primed to an HPIV3, HPIV1 or other HPIV peptide epitope and/or expanded, for example, to increase the numbers of HPIV-specific T cells. In some embodiments, the at least one isolated subpopulation is derived from cord blood, from precursor T cells, or stem cells capable of differentiation into T cells.

The composition may comprise subpopulation(s) of T cells recognizing one, two, three, four, five or more peptide epitopes contained in Peptide 84, Peptide 83, Peptide 82, Peptide 59, Peptide 85, Peptide 60, Peptide 76, Peptide 78, Peptide 38, Peptide 77, Peptide 39 or Peptide 50 or peptide variants of these specific peptides. In another embodiment, the subpopulation(s) of T cells recognize more than one, two, three, four, or five peptide epitopes contained in any one of Peptide 84, Peptide 83, Peptide 82, Peptide 59, Peptide 85, Peptide 60, Peptide 76, Peptide 78, Peptide 38, Peptide 77, Peptide 39 or Peptide 50, and the at least one isolated subpopulation of T cells recognizes an epitope contained in Peptide 84, 83 or 82.

Another aspect of the disclosure is directed to a virus-specific (VST) T cell bank comprising the compositions described above, wherein said composition comprises a single subpopulation of T cells recognizing a peptide antigen to HPIV3, HPIV1 or other HPIV or a plurality of said subpopulations and wherein said bank optionally describes or categorizes the HLA restriction of said subpopulation(s). Typically, each subpopulation recognizes a particular T cell peptide epitope, though in some embodiments there may be cross-reactivity among HPIV specific T cells.

Another aspect of the invention is directed to a composition which comprises at least one peptide containing an epitope of Peptide 84, Peptide 83, Peptide 82, Peptide 59, Peptide 85, Peptide 60, Peptide 76, Peptide 78, Peptide 38, Peptide 77, Peptide 39 or Peptide 50; or comprises at least one peptide containing an epitope of HPIV3, HPIV1 or other HPIV that comprises one, two, three, four or more insertions, substitutions, or deletions to an amino acid sequence of Peptide 84, Peptide 83, Peptide 82, Peptide 59, Peptide 85, Peptide 60, Peptide 76, Peptide 78, Peptide 38, Peptide 77, Peptide 39 or Peptide 50 In some embodiments, the composition comprises at least one, two, three, four, five or more of said peptides and comprises at least one peptide containing an epitope of Peptide 84, 83 or 82 which data shows are immunodominant. In some embodiments, the peptide compositions may further comprise an adjuvant, especially adjuvants which help prime and expand T cell subpopulations that recognize HLA-restricted peptides disclosed herein.

Another aspect of the invention is directed to method for priming or expanding in vivo or ex vivo at least one subpopulation of T cells recognizing HPIV3, HPIV1 or other HPIV epitopes comprising contacting a subject at least one of Peptide 84, Peptide 83, Peptide 82, Peptide 59, Peptide 85, Peptide 60, Peptide 76, Peptide 78, Peptide 38, Peptide 77, Peptide 39 or Peptide 50. Such a method may comprise use of at least one immunodominant HPIV3, HPIV1 or other HPIV epitope contained on Peptide 84, 83 or 82.

In another embodiment, this method involves administering the peptides or peptides and adjuvants intravenously, intramuscularly, subcutaneous, orally, or via the nose, the upper or lower respiratory system, for example, by inhalation or dispersion of the peptide composition into an aerosol or powder.

In some embodiments, the method further comprises administering an adjuvant prior to, during, or after said immunizing.

Another aspect of the invention involves a method for isolating a subpopulation of T cells that recognize HLA restricted peptide epitopes of HPIV3 comprising contacting T cells with an overlapping peptide library of at least one HPIV3 in the presence of dendritic cells or other antigen presenting cells, and isolating T cells which recognize one or more peptides in said overlapping peptide library as presented by the antigen presenting cells. This method may further comprise contacting said T cells in combination with the overlapping peptide library with IL-4 and IL-7 or other cytokines known in the art for facilitating priming or expansion of T cells or pre-T cells. In one embodiment, the overlapping peptide library is for HPIV3, HPIV1 or other HPIV matrix protein and the isolated T cells recognize a peptide epitope contained within Peptide 84, Peptide 83, Peptide 82, Peptide 59, Peptide 85, Peptide 60, Peptide 76, Peptide 78, Peptide 38, Peptide 77, Peptide 39 or Peptide 50 or their variants as disclosed herein.

The invention also involves a method for using the HPIV-specific T cells to produce or manufacture a therapeutic T cell composition suitable for adoptive transfer to a subject in need thereof or to a method for using the isolated HPIV peptides disclosed herein to produce an immunogenic composition or vaccine for administration to a subject in need thereof. Such embodiments comprise a process of manufacture of such a HPIV-specific T cell composition or immunogenic peptide composition for the purpose of the new therapeutic use for prevention or treatment of HPIV infection or for generation of cellular immunity to HPIV.

Also, provided herein are compositions and methods for use in T-cell therapy to treat HPIV3 infections. Non-engineered T-cell compositions that include in the same dosage of one or a plurality of T-cell subpopulations are provided for administration to a human patient with HPIV3 infection, wherein each T-cell subpopulation is primed with one or more HPIV3 antigens disclosed herein, and the T-cell subpopulations comprised in the T-cell composition for administration are chosen specifically based on the HPIV3 immunodominant antigens detected in a subject infected with HPIV3. By using separate activated T-cell subpopulations to form the T-cell composition for administration, the T-cell composition as a whole includes individual T-cell subpopulations targeting HPIV3-specific antigens, resulting in a highly consistent and activated T-cell composition capable of targeting cells infected with HPIV3.

In one aspect, the present disclosure relates to an isolated T-cell composition comprising one or a plurality of T-cell subpopulations, each T-cell subpopulation is specific for a single human parainfluenza virus-3 (HPIV3) antigen or epitope. In some embodiments, the single HPIV3 antigen is chosen from the hemagglutinin-neuramidase glycoprotein (HN), the fusion protein (Fus), the structural matrix protein (Mat), the nucleocapsid protein (NP), the P phosphoprotein (P), and the L protein (L), or functional fragments thereof. In other embodiments, each T-cell subpopulation is primed and expanded separately from each other. In some embodiments, each T-cell subpopulation is primed and expanded ex vivo. In some embodiments, one or more of the T-cell subpopulations is originated from umbilical cord blood.

In some embodiments, each T-cell subpopulation is primed and expanded using a group of peptides comprising peptides specific to each HPIV3 antigen that are HLA-restricted to one or more HLA alleles of the donor cell source. In some embodiments, each of the T-cell subpopulations is primed and expanded using a group of peptides comprising peptides specific to each HPIV3 antigen that are HLA-restricted to one or more HLA alleles of the donor cell source, wherein the one or more HLA alleles are selected from HLA-A, HLA-B, and HLA-C. In some embodiments, each of the T-cell subpopulations is primed and expanded using a group of peptides comprising peptides specific to each HPIV3 antigen that are HLA-restricted to at least one of the donor's HLA-A alleles, at least one of the donor's HLA-B allele, and at least one of the donor's HLA-DR alleles. In some embodiments, each of the T-cell subpopulations is primed and expanded using a group of peptides comprising peptides specific to each HPIV3 antigen that are HLA-restricted to at least both of the donor's HLA-A alleles, at least both of the donor's HLA-B allele, and at least both of the donor's HLA-DR alleles. In some embodiments, the HLA-A allele is HLA-A*01, HLA-A*02:01, HLA-A*03, HLA-A*11:01, HLA-A*24:02, HLA-A*26, or HLA-A*68:01. In some embodiments, the HLA-B allele is HLA-B*07:02, HLA-B*08, HLA-B*15:01 (B62), HLA-B*18, HLA-B*27:05, HLA-B*35:01, or HLA-B*58:02. In some embodiments, the HLA-DR allele is HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b).

In some embodiments, at least one T-cell subpopulation is specific for the HN of HPIV3, or a functional fragment thereof. In some embodiments, at least one T-cell subpopulation is specific for the Fus of HPIV3, or a functional fragment thereof. In some embodiments, at least one T-cell subpopulation is specific for the Mat of HPIV3, or a functional fragment thereof. In some embodiments, at least one T-cell subpopulation is specific for the NP of HPIV3, or a functional fragment thereof. In some embodiments, at least one T-cell subpopulation is specific for the P of HPIV3, or a functional fragment thereof. In some embodiments, at least one T-cell subpopulation is specific for the L of HPIV3, or a functional fragment thereof.

In some embodiments, each T-cell subpopulation is combined in the disclosed T-cell composition in a defined ratio, wherein the defined ratio is based on either total cell number or normalized cell activity. In some embodiments, the disclosed T-cell composition consists of from about one, two, three, four, five to about six T-cell subpopulations. In some embodiments, the disclosed T-cell composition comprises at least about 45% of a first T-cell subpopulation, at least about 10% of a second T-cell subpopulation, at least about 5% of a third T-cell subpopulation, and at least about 5% of a fourth T-cell subpopulation. In some embodiments, the single HPIV3 antigen used to prime the T-cell subpopulation comprises an epitope comprising at least 75%, 85%, 90%, 95%, 99% or <100% sequence identity to SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19.

BLASTP can be used to identify an amino acid sequence having at least 70%, 75%, 80%, 85%, 87.5%, 90%, 92.5%, 95%, 97.5%, 98%, 99% or <100% sequence identity, or similarity to a reference amino acid, such as an HPIV amino acid sequence, using a similarity matrix such as BLOSUM45, BLOSUM62 or BLOSUM80 where BLOSUM45 can be used for closely related sequences, BLOSUM62 for midrange sequences, and BLOSUM80 for more distantly related sequences. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity or similarity to the amino acid sequences disclosed herein are contemplated and encompassed by this disclosure. A representative BLASTP setting that uses an Expect Threshold of 10, a Word Size of 3, BLOSUM 62 as a matrix, and Gap Penalty of 11 (Existence) and 1 (Extension) and a conditional compositional score matrix adjustment. Other default settings for BLASTP are described by and incorporated by reference to the disclosure available at: hypertext transfer protocol secure://blast.ncbi.nlm.nih.gov/Blast.cgi?PROGRAM-blastp&PAGE_TYPE=BlastSearch&LINK_LOC=blasthome (last accessed Dec. 9, 2020). A variant of the proteins described by sequence identifier herein may also comprise 1, 2, 3, 4, 5, 6, 7, 8, 9, 10, or >10 amino acid deletions, substitutions, or insertions compared to a reference sequence.

In some embodiments, the single HPIV3 antigen used to prime the T-cell subpopulation comprises an epitope comprising at least 85% sequence identity to SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO. 15, SEQ ID NO: 16, SEQ ID NO: 17, SEQ ID NO: 18 or SEQ ID NO: 19. In some embodiments, the single HPIV3 antigen used to prime the T-cell subpopulation comprises an epitope comprising at least 95% sequence identity to SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19. In some embodiments, the single HPIV3 antigen used to prime the T-cell subpopulation comprises an epitope comprising the amino acid sequence of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19.

In another aspect, the present disclosure related to a pharmaceutical composition comprising any of the isolated T-cell composition disclosed herein and a pharmaceutically acceptable carrier.

In a further aspect, the present disclosure relates to a method of treating HPIV3 infection in a subject in need thereof. In some embodiments, the method comprises administering a therapeutically effective amount of any of the isolated T-cell composition disclosed herein or a pharmaceutical composition comprising such an isolated T-cell composition to the subject in need thereof. In some embodiments, the subject in need thereof suffers acute respiratory infections and/or is immunodeficiency due to genetic disorders, solid organ transplantation, or hematopoietic stem cell transplant (HSCT). In some embodiments, the isolated T-cell composition has at least one HLA allele or HLA allele combination in common with the subject. In some embodiments, the isolated T-cell composition has more than one HLA allele or HLA allele combination in common with the subject. In some embodiments, the administration comprises administering a first dose followed by at least one additional dose, wherein the additional dose is administered at an interval selected from about 1 week, about 2 weeks, about 3 weeks, about 4 weeks, about 5 weeks, about 6 weeks, about 7 weeks or about 8 weeks. In some embodiments, the therapeutically effective amount of cells results in more than about 1 log-fold reduction of detectable HPIV3 genome copies or detection of expansion of HPIV3-specific T-cells as measured by ELISpot.

In some embodiments, the method of treating a subject with a HPIV3 infection comprises
i) determining an HLA subtype of the subject;
ii) identifying one or a plurality of HPIV3 antigens associated with a tissue or cell of the subject for targeting;
iii) selecting one or a plurality of banked T-cell subpopulations having the highest activity against the one or plurality of HPIV3 antigens through one or more HLA-alleles shared between the subject and the T-cell subpopulation, wherein at least one T-cell subpopulation is specific for a single HPIV3 antigen;
iv) combining the one or plurality of banked T-cell subpopulations selected in step iii) to create a T-cell composition; and
v) administering a therapeutically effective amount of the T-cell composition to the subject.

In some embodiments, each T-cell subpopulation is primed and expanded separately from each other ex vivo. In some embodiments, the single HPIV3 antigen is chosen from the hemagglutinin-neuramidase glycoprotein, the fusion protein, the structural matrix protein, the nucleocapsid protein, the P phosphoprotein, and the L protein of HPIV3, or functional fragments thereof. In some embodiments, at least one of the T-cell subpopulations selected in step iii) is primed and expanded by one or more HLA-restricted peptides selected from Table 1.

In some embodiments, the method of treating a patient with a HPIV3 infection comprises:
i) determining an HLA subtype of the patient;
ii) determining the HPIV3 antigen expression profile of the patient's HPIV3;
iii) identifying two or more HPIV3 antigens expressed by the patient's HPIV3 for targeting;
iv) selecting one banked T-cell subpopulation having the highest activity against each targeted HPIV3 antigen through one or more HLA-alleles shared between the patient and the T-cell subpopulations, wherein at least one T-cell subpopulation is specific for a single HPIV3 antigen, wherein each T-cell subpopulation is specific for a different HPIV3 antigen or a different combination of HPIV3 antigens, wherein each T-cell subpopulation is primed and expanded separately from each other, and wherein each T-cell subpopulation is expanded ex vivo;
v) combining each selected banked T-cell subpopulation to create a T-cell composition; and
vi) administering a therapeutically effective amount of the T-cell composition to the patient.

In some embodiments, the single HPIV3 antigen is chosen from the hemagglutinin-neuramidase glycoprotein, the fusion protein, the structural matrix protein, the nucleocapsid protein, the P phosphoprotein, and the L protein of HPIV3, or functional fragments thereof. In some embodiments, at least one banked T-cell subpopulation selected in step iv) is primed and expanded by one or more HLA-restricted peptides selected from Table 1.

In some embodiments, the method of treating a patient with a HPIV3 infection comprises:
i) determining an HLA subtype of the patient;
ii) determining the HPIV3 antigen expression profile of the patient's HPIV3;
iii) identifying two or more HPIV3 antigens expressed by the patient's HPIV3 for targeting;
iv) selecting one banked T-cell subpopulation having the highest activity against each targeted HPIV3 antigen through one or more HLA-alleles shared between the patient and the T-cell subpopulations, wherein at least one T-cell subpopulation is specific for a single HPIV3 antigen, wherein each T-cell subpopulation is specific for a different HPIV3 antigen or a different combination of HPIV3 antigens, wherein each T-cell subpopulation is primed and expanded separately from each other, and wherein each T-cell subpopulation is primed and expanded ex vivo;
v) combining each selected banked T-cell subpopulation to create a first T-cell composition;
vi) administering a therapeutically effective amount of the first T-cell composition to the patient;
vii) monitoring the patient's response to the first T-cell composition by measuring the presence of circulating HPIV3 antigen specific T-cells;
viii) monitoring changes to the patient's HPIV3 antigen expression profile;
ix) if the patient's HPIV3 antigen expression profile has changed, identifying two or more HPIV3 antigens expressed by the patient's HPIV3 for targeting, wherein if the patient is showing a robust response to any specific HPIV3 antigen T-cell subpopulation(s) from the first T-cell composition, exclude targeting that HPIV3 antigen;
x) selecting one banked T-cell subpopulation having the highest activity against each targeted HPIV3 antigen selected from step ix) through one or more HLA-alleles shared between the patient and the T-cell subpopulations, wherein at least one T-cell subpopulation is specific for a single HPIV3 antigen, wherein each T-cell subpopulation is specific for a different HPIV3 antigen or a different combination of HPIV3 antigens, wherein each T-cell subpopulation is primed and expanded separately from each other, and wherein each T-cell subpopulation is primed and expanded ex vivo;
xi) combining each selected banked T-cell subpopulation to create a second T-cell composition; and
xii) administering a therapeutically effective amount of the second T-cell composition to the patient; and optionally,
xiii) repeating steps vii) to x),
xiv) combining each selected banked T-cell subpopulation to create a third T-cell composition; and
xv) administering a therapeutically effective amount of the third T-cell composition to the patient.

In some embodiments, the single HPIV3 antigen is chosen from the hemagglutinin-neuramidase glycoprotein, the fusion protein, the structural matrix protein, the nucleocapsid protein, the P phosphoprotein, and the L protein of HPIV3, or functional fragments thereof. In some embodiments, at least one banked T-cell subpopulation selected in step iv) and/or step x) is primed and expanded by one or more HLA-restricted peptides selected from Table 1.

In some embodiments, the method of treating a patient with a HPIV3 infection comprises.
i) generating one or more HPIV3 specific T-cell subpopulations from the patient or a healthy relative of the patient, wherein at least one T-cell subpopulation is specific for a single HPIV3 antigen, wherein each T-cell subpopulation is specific for a different HPIV3 antigen or a different combination of HPIV3 antigens, wherein each T-cell subpopulation is primed and expanded separately from each other, and wherein each T-cell subpopulation is expanded ex vivo;
ii) combining each T-cell subpopulation to create a T-cell composition; and
iii) administering a therapeutically effective amount of the T-cell composition to the patient.

In some embodiments, the single HPIV3 antigen is chosen from the hemagglutinin-neuramidase glycoprotein, the fusion protein, the structural matrix protein, the nucleocapsid protein, the P phosphoprotein, and the L protein of HPIV3, or functional fragments thereof. In some embodiments, at least one T-cell subpopulation is primed and expanded by one or more HLA-restricted peptides selected from Table 1.

In yet another aspect, the present disclosure relates to a library of isolated T-cell subpopulations comprising two or more characterized T-cell subpopulations, wherein each T-cell subpopulation is originated from an allogeneic donor; wherein each T-cell subpopulation is specific for a single HPIV3 antigen; wherein each T-cell subpopulation is primed and expanded separately from each other; wherein each T-cell subpopulation is primed and expanded ex vivo; wherein each T-cell subpopulation is characterized or identified by:
i) HLA-phenotype;
ii) specificity to the single HPIV3 antigen;
iii) epitope or epitopes each T-cell subpopulation is specific to,
iv) MHC Class each T-cell subpopulation is restricted to;
v) antigenic activity through the T-cell's corresponding HLA-allele; and
vi) immune effector subtype concentration;
   and wherein the characterization of each T-cell subpopulation is recorded in a database for future reference, and the T-cell subpopulations are cryopreserved for future use. In some embodiments, at least one T-cell subpopulation is primed and expanded by one or more HLA-restricted peptides selected from Table 1.

In some embodiments, one or more T-cell subpopulations used in any of the methods disclosed herein are primed and expanded with an overlapping peptide library.

In another aspect, the present disclosure provides a method of treating HPIV1 infection in a subject in need thereof comprising administering a therapeutically effective amount of any of the isolated T-cell compositions disclosed herein or any of the pharmaceutical compositions comprising such T-cell compositions to the subject in need thereof. In some embodiments, the isolated T-cell composition comprises at least one T-cell subpopulation that is specific for a single HPIV3 antigen and cross-reacts with at least one HPIV1 antigen. In some embodiments, the isolated T-cell composition has at least one HLA allele or HLA allele combination in common with the subject. In some embodiments, the isolated T-cell composition has more than one HLA allele or HLA allele combination in common with the subject. In some embodiments, the administration comprises administering a first dose followed by at least one additional dose, wherein the additional dose is administered at an interval selected from about 1 week, about 2 weeks, about 3 weeks, about 4 weeks, about 5 weeks, about 6 weeks, about 7 weeks or about 8 weeks. In some embodiments, the therapeutically effective amount of cells results in more than about 1 log-fold reduction of detectable HPIV1 genome copies in the subject.

In yet another aspect, the present disclosure provides a method of treating a subject with a HPIV1 infection comprising:
i) determining an HLA subtype of the subject;
ii) identifying one or a plurality of HPIV1 antigens associated with a tissue or cell of the subject for targeting;
iii) selecting one or a plurality of banked HPIV3-specific T-cell subpopulations based on sequence homology between the one or plurality of HPIV1 antigens and the HPIV3 antigens used for generating the banked HPIV3-specific T-cell subpopulations and through one or more HLA-alleles shared between the subject and the T-cell subpopulation, wherein at least one banked HPIV3-specific T-cell subpopulation is specific for a single HPIV3 antigen, and wherein the sequence homology is at least about 50% sequence identity;
iv) combining the one or plurality of banked T-cell subpopulations selected in step iii) to create a T-cell composition; and
v) administering a therapeutically effective amount of the T-cell composition to the subject.

In some embodiments, each of the HPIV3-specific T-cell subpopulations is primed and/or expanded separately from each other ex vivo. In some embodiments, the single HPIV3 antigen is chosen from hemagglutinin-neuramidase glycoprotein, fusion protein, structural matrix protein, nucleocapsid protein, P phosphoprotein, and L protein, or functional fragments thereof. In some embodiments, the sequence homology between the one or plurality of HPIV1 antigens and the HPIV3 antigens used for generating the banked HPIV3-specific T-cell subpopulations is at least about 80% sequence identity. In some embodiments, at least one of the T-cell subpopulations selected in step iii) is primed and expanded by one or more HLA-restricted peptides selected from Table 1.

### BRIEF DESCRIPTION OF THE DRAWINGS

FIG. 1 depicts generation of T-cell subpopulations from peripheral blood using HPIV3 pepmixes, IL-4 and IL-7.

FIG. 2 shows confirmation of HLA restriction, administration of partially HLA-matched HPIV3-VSTs, and timing of infusion(s) of HPIV3-VSTs, to resolve HPIV3 infection.

FIG. 3 is an example chart and not from a specific patient. Many patient responses to administration of HPIV3-VSTs during symptomatic HPIV3 infection and subsequent levels of immune responses (spots per well, SPW) after a VST infusion resemble the graph on this chart.

FIG. 4A. Virus-specific T-cells (VST) Product Phenotyping using flow cytometry.

FIG. 4B. Overall HPIV3 Matrix specificity by product. All 10 viral-specific T cell products were stimulated with HPIV3 pepmix. Response were measured as spot forming units per well (SFU/1×10⁵ cells) by anti-LFN-g ELISpot assay. Unstimulated T-cells (CTL only) and stimulation with actin pepmix (irrelevant antigen) were used as negative controls.

FIG. 5. Peptide Pool specificity by product. Virus-specific T-cells (VSTs) were stimulated with combinatorial HPIV3 peptide pools. Response was measured as spots per well (SFU/1×10⁵ cells) by anti-IFN-g ELISpot assay. Values from unstimulated T-cells (CTL only) and stimulation of cells with actin (an irrelevant antigen) were used as negative controls.

FIG. 6A. Cross reactivity between HPIV3 and HPIV1. Virus-specific T-cells (VSTs) were stimulated with individual HPIV3 peptides and corresponding HPIV1 peptides. Response was measured as spots per well (SFU/1×10⁵ cells) by anti-IFN-g ELISpot assay, and mean responses were calculated among responding products. Black bars (upper bars) represent SFU values of VSTs to HPIV3 peptides and gray bars (lower bars) represent SFU values of V HPIV1 peptides.

FIG. 6B. CD4+ T Cell Responses against HPIV3 and HPIV1 peptides. Intracellular flow cytometry was performed on VSTs following stimulation with Actin, SEB, HPIV3 or HPIV1 peptides and anti-CD28/CD49. After 2 h stimulation with peptides, Brefeldin A was added for an additional 4 h following which cells were labeled with dead cell exclusion dye, Fc Receptor block and antibodies against CD3, CD4, CD8, TNF-α, IFN-g, and CD95. Following fixation and permeabilization cells were labeled intracellularly with antibodies targeting TNF-α, IFN-g, Percentage of CD4+ and CD8+ T-cells producing both IFN-gamma+ and/or TNFalpha+ were measured.

FIG. 7A. HLA Restriction Mapping. Two phytohemagglutinin (PHA) blast lines (each partially matched to a Virus-specific T-cell (VST) product at HLA-DR*15:01 and HLA-DQ*03:01, respectively) were pulsed with HPIV3 peptides 82, 83, and 84 for 60 min, triple washed, and then presented to VSTs. T cell activity was detected using anti-IFN-g ELISpot.

FIG. 7B. HPIV3 Matrix diagram with epitopes and C-terminal domain. HPIV3 Matrix protein contains 353 amino acids. Diamonds represent the novel epitopes identified during mapping. The oligomerization domain at the C-terminus contains 7 out of 12 epitopes, including the three most immunodominant peptides in Peptides 82-84. The black arrow denotes the L302 residue, which is a target of ubiquitination during virion production.

### DETAILED DESCRIPTION OF EMBODIMENTS

Provided herein are adoptive T-cell therapies to treat human parainfluenza virus-3 (HPIV3) infections which include administering to a subject in need thereof a therapeutically effective amount of a T-cell composition that includes in the same dosage form one or a plurality of T-cell subpopulations, wherein each T-cell subpopulation is specific for one or a plurality of HPIV3 antigens. In some embodiments, the T-cell subpopulations comprised in the T-cell composition for administration are chosen specifically based on the HPIV3 antigen profile of the virus found in the subject.

Further, importantly, this advantageous T-cell therapy can be optimized for personal efficacy in the subject by maximizing cross-reactivity of included viral epitopes, thus improving the breadth of clinical viral strains targeted by the T-cells.

The present disclosure can be understood more readily by reference to the following detailed description of embodiments, the figures and the examples included herein.

### Definitions

Unless defined otherwise, all technical and scientific terms used herein have the same meaning as commonly understood by one of ordinary skill in the art to which the disclosure pertains.

The term “a” and “an” refers to one or to more than one (i.e., to at least one) of the grammatical object of the article. By way of example, “an element” means one element or more than one element.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, 0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

The term “adjuvant” refers to a pharmacological or immunological agent that improves the immune response of a vaccine, such as to a peptide comprising an epitope of HPIV3 as disclosed herein. Useful antigens for boosting cellular immune responses to peptides are known in the art an include complete Freund's adjuvant (CFA), incomplete Freund's adjuvant (IFA), alumina adjuvants such as aluminum hydroxide or aluminum phosphate, micro/nanoparticles, antigen/peptide depot administration, immunopotentiators, adjuvants targeting toll-like receptors including TLR2, TLR3, TLR 4, TLF7/8 and TLR9 agonists, STING agonists, cytokines such as IL-2, GM-CSF, and interferons, liposomes, detergents such as Quil A or saponin, and bacterial products such as lipopolysaccharide, killed B. periussis or mycobacterium, or toxoids. These and other adjuvants are incorporated by reference to Khong, H. & Overwijk, W. W, Adjuvants for peptide-base cancer vaccines, J. IMMUNOTHERAPY OF CANCER, 2016, 4, 56 and to those used by commercial adjuvant services, such as those described by Creative Biolabs hypertext transfer protocol secure://www.creative-biolabs.com/vaccine/vaccine-technology.htm (last accessed Dec. 7, 2020, incorporated by reference).

The term “allogeneic” as used herein refers to medical therapy in which the donor and recipient are different individuals of the same species who do not have identical genetic backgrounds.

The term “antigen” as used herein refers to molecules, such as polypeptides, peptides, or glyco- or lipo-peptides that are recognized by the immune system, such as by the cellular or humoral arms of the human immune system. The term “antigen” includes antigenic determinants, such as peptides with lengths of 6, 7, 8, 9, 10, 11, 12, 13, 14, 15, 16, 17, 18, 19, 20, 21, 22 or more amino acid residues that bind to major histocompatibility complex (MHC) molecules, form parts of MHC Class I or II complexes, or that are recognized when complexed with such molecules.

The term “antigen presenting cell” (APC) as used herein refers to a class of cells capable of presenting one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen.

The term “autologous” as used herein refers to medical therapy in which the donor and recipient are the same person or have identical HLA backgrounds (e.g. identical twins).

The term “cord blood” as used herein has its normal meaning in the art and refers to blood that remains in the placenta and umbilical cord after birth and contains hematopoietic stem cells. Cord blood may be fresh, cryopreserved, or obtained from a cord blood bank.

The term “cytokine” as used herein has its normal meaning in the art. Nonlimiting examples of cytokines used in the disclosure include interleukin-2 (IL-2), IL-4, IL-6, IL-7, IL-12, IL-15, and IL-27.

The term “cytotoxic T-cell” or “cytotoxic T lymphocyte” as used herein is a type of immune cell that bears a CD8+ antigen and that can kill certain cells, including foreign cells, tumor cells, and cells infected with a virus. Cytotoxic T-cells can be separated from other blood cells, grown ex vivo, and then given to a patient to kill tumor or viral cells. A cytotoxic T-cell is a type of white blood cell and a type of lymphocyte.

The term “dendritic cell” or “DC” as used herein describes a diverse population of morphologically similar cell types found in a variety of lymphoid and non-lymphoid tissues, see Steinman, Ann. Rev. Immunol. 9:271-296 (1991).

The term “effector cell” as used herein describes a cell that can bind to or otherwise recognize an antigen and mediate an immune response. Tumor-, virus-, or other antigen-specific T-cells and NKT-cells are examples of effector cells.

The term “endogenous” as used herein refers to any material from or produced inside an organism, cell, tissue or system.

The term “epitope” or “antigenic determinant” as used herein refers to the part of an antigen that is recognized by the immune system, specifically by antibodies, B cells, or T cells.

The term “exogenous” as used herein refers to any material introduced from or produced outside an organism, cell, tissue or system.

The term “functional fragment” as used herein refers to a portion of the full-length of a HPIV3 antigen, such as HN, Fus, Mat, NP, P or L, that retains some or all of the activity (e.g., biological activity, HLA binding, T cell binding) of the full-length polypeptide. Such functional fragments can be any size, provided that the fragment retains some or all of the activity of the full-length polypeptide. For example, a functional fragment of HN, Fus, Mat, NP, P or L can be, for example, about 10, 20, 30, 40, 50, 60, 70, 80, 90, 100, 150, 200, 300, 350, etc., amino acids in length. A “functional fragment” as used herein can also be a fragment that comprises at least about 70%, 75%, 80%, 85%, 90%, 95%, 96%, 97%, 98%, or 99% sequence identity to HN, Fus, Mat, NP, P or L, provided that such fragments retains some or all of the activity (e.g., biological activity) of the full-length polypeptide.

A homolog of a peptide, such as the peptides disclosed herein containing T cell epitopes, may share at least 7, 8, 9, 10, 11, 12, 13, 14 or 15 amino acid residues with a reference peptide. For example, the HPIV1 homolog of Peptide 84 (SEQ ID NO: 3) shares 10 amino acid residues with Peptide 84 (SEQ ID NO: 18). Other homologs of Peptide 84 may share fewer or more amino acid residues with Peptide 84 (SEQ ID NO: 18).

The term “HLA” as used herein refers to human leukocyte antigen. There are 7,196 known HLA alleles. These are divided into 6 HLA class I and 6 HLA class II alleles for each individual (on two chromosomes). The HLA system or complex is a gene complex encoding the major histocompatibility complex (MHC) proteins in humans. HLAs corresponding to MHC Class I (A, B, or C) present peptides from within the cell and activate CD8-positive (i.e., cytotoxic) T-cells. HLAs corresponding to MHC Class II (DP, DM, DOA, DOB, DQ and DR) stimulate the multiplication of CD4-positive T-cells which stimulate antibody-producing B-cells.

The term “isolated” as used herein means separated from components in which a material is ordinarily associated with, for example, an isolated cord blood mononuclear cell can be separated from red blood cells, plasma, and other components of cord blood, or PBMCs or T-cells can be separated from red blood cells and other humoral or cellular components of blood. T cells may also be isolated by T cell cloning and/or expansion of primed T cells, for example, as described by Bollard, et al., U.S. 2018/0072990A1 (incorporated by reference) or by S. Mariotti & R. Nisini, Generation of human T cell clones, T CELL PROTOCOLS, pp. 65-93 (incorporated by reference) or by other methods known in the art. The term “isolated” also includes selective enrichment of particular T cell clones, such as the enrichment of T cells recognizing HLA restricted HPIV3 peptide antigens compared to a level in a control, unenriched T cell sample. Enrichment includes increasing the number of T cells recognizing an HLA restricted antigen by 10, 20, 50, 100, 200, 500% or more compared to the number of T cells recognizing the HLA restricted antigen in a control sample, such as a subject not receiving an adoptive transfer of T cells or a subject who has not been immunized with restricted peptide epitopes recognized by a subject's T cells.

A “naïve” T-cell or other immune effector cell as used herein is one that has not been exposed to or primed by an antigen or to an antigen-presenting cell presenting a peptide antigen capable of activating that cell. In some instances, a subject's immune system will be naïve to a particular epitope in other instances some of a subject's T-cells or immune effector cells will be naïve to a particular antigen. Naïve T cells often express cell surface markers such as CD45RA, CCR7, CD62L, CD127, or CD132 or can lack expression of markers of previous activation, such as CD25, CD44, CD69, CD45RO, or HLA-DR.

The term “non-engineered” when referring to the cells of the compositions means a cell that does not contain or express an exogenous nucleic acid or amino acid sequence. For example, the cells of the compositions do not express, for example, a chimeric antigen receptor. In other embodiments, the term “non-engineered” refers to a cell which has not be transfected or transformed with a particular exogenous nucleic acid.

The term “originated from” as used herein refers to the origin from which the primary T-cells used for expansion and preparation of any of the T-cell subpopulations disclosed herein are obtained. For instance, in some embodiments, one or more of the T-cell subpopulations is originated from umbilical cord blood, which means that the primary T-cells used for expansion and preparation of those T-cell subpopulations are obtained from umbilical cord blood. Likewise, in some embodiments, each T-cell subpopulation comprised in the library of isolated T-cell subpopulations according to the present disclosure are originated from an allogeneic donor; which means that the primary T-cells used for expansion and preparation of each T-cell subpopulation are obtained from an allogeneic donor.

Similarly, when referring to the mononuclear cell sample from which the T-cell subpopulations are isolated, the term “originated from” refers to the origin from which the mononuclear cell sample is obtained. For example, in some embodiments, the mononuclear cell sample is originated from the same human subject to which the disclosed composition is also administered (autologous), which means that the mononuclear cell sample is obtained from the autologous human subject. Likewise, in some embodiments, the mononuclear cell sample is originated from a cell donor (e.g., an allogeneic donor), which means that the mononuclear cell sample is obtained from an allogeneic donor.

A “peptide library” or “overlapping peptide library” as used herein within the meaning of the application is a complex mixture of peptides which in the aggregate covers the partial or complete sequence of a protein antigen. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries are commercially available or may be custom-made for particular antigens.

A “peripheral blood mononuclear cell” or “PBMC” as used herein is any peripheral blood cell having a round nucleus. These cells consist of lymphocytes (T-cells, B-cells, NK cells) and monocytes. In humans, lymphocytes make up the majority of the PBMC population, followed by monocytes, and a small percentage of dendritic cells.

The term “precursor cell” as used herein refers to a cell which can differentiate or otherwise be transformed into a particular kind of cell. For example, a “T-cell precursor cell” can differentiate into a T-cell and a “dendritic precursor cell” can differentiate into a dendritic cell.

A “subject” or “host” as used herein is a vertebrate, preferably a mammal, more preferably a human. Mammals include, but are not limited to humans, simians, equines, bovines, porcines, canines, felines, murines, other farm animals, sport animals, or pets. Typically, the method as directed to non-human animals involves identification of homologs of the T cell epitopes disclosed herein and identification of MHC restriction suitable for recognition of these epitopes by HPIV-specific T cells in an animal.

A “patient” or “subject in need thereof” as used herein includes those in need of virus- or other antigen-specific T-cells, such as those with lymphocytopenia, those who have undergone immune system ablation, those with primary immunodeficiency disorders, those undergoing transplantation and/or immunosuppressive regimens, those having naïve or developing immune systems, such as neonates, or those undergoing cord blood or stem cell transplantation. This term also includes those at risk of exposure to, or at risk of infection by HPIV including HPIV1 and HPIV3. In a typical embodiment, the term “patient” or “subject in need thereof” as used herein refers to a human.

A “T-cell population” or “T-cell subpopulation” can include thymocytes, immature T-lymphocytes, mature T-lymphocytes, resting T-lymphocytes and activated T-lymphocytes. The T-cell population or subpopulation can include as' T-cells, including CD4+ T-cells, CD8+ T cells, γδ T-cells, Natural Killer T-cells, or any other subset of T-cells. In some embodiments, a T cell subpopulation will recognize a particular peptide antigen, peptide epitope, or combination of an HLA molecule and peptide epitope.

The term “disclosed composition” refers to a T-cell composition comprising one or a plurality of T-cell subpopulations. The disclosed composition is comprised of one or more T-cell subpopulations, wherein at least one T-cell subpopulation targets a single HPIV3 antigen. For purposes herein, when referring to combining T-cell subpopulations to compose the disclosed composition, combining is intended to include the situation wherein the T-cell subpopulations are physically combined into a single dosage form, that is, a single composition. In alternative embodiments, the T-cell subpopulations are kept physically separated but administrated concomitantly and collectively comprise the disclosed composition.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. In some embodiments, the disclosed compositions are administered with at least one pharmaceutically acceptable carrier.

The term “pharmaceutically acceptable carrier” is art recognized and includes a pharmaceutically acceptable material, composition or vehicle, suitable for administering compositions of the present disclosure to subjects. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but not limited to, sugars, such as lactose, glucose and sucrose, starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar, buffering agents, such as magnesium hydroxide and aluminum hydroxide; alginic acid; pyrogen-free water, isotonic saline, Ringer's solution; ethyl alcohol, phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. In some embodiments, the pharmaceutically acceptable carrier is sterile and pyrogen-free water. In some embodiments, the pharmaceutically acceptable carrier is Ringer's Lactate, sometimes known as lactated Ringer's solution.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

HPIV3 Antigens. T-cell subpopulations targeting one or more HPIV3 antigens can be prepared by pulsing antigen presenting cells or artificial antigen presenting cells with one or more peptides or epitopes from HPIV3. In some embodiments, if more than one peptide from a single HPIV3 antigen is used, the peptide segments can be generated by making overlapping peptide fragments of the HPIV3 antigen, as provided for example in commercially available overlapping peptide libraries or “PepMix™.” In certain aspect, the overlapping peptide libraries include peptides that are about 7, 8, 9, 10, 11, 12, 13, 14, 15, 16 or more amino acids long and overlapping one another by about 5, 6, 7, 8, 9, 10, 11 or more amino acids. In particular embodiments, the peptides are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length. In some embodiments, the overlapping peptide fragments can be further enriched with certain antigenic epitopes of the targeted HPIV3 antigen that are active through specific cell donor human leukocyte antigen (HLA) alleles, for example, a single specific HLA-restricted epitope or multiple specific HLA-restricted epitopes of HPIV3. In certain embodiments, if more than one peptide from a single, targeted HPIV3 antigen or a plurality of HPIV3 antigens are used, the peptide fragments can be selected from certain antigenic epitopes of the targeted HPIV3 antigen that are active through specific HLA alleles, for example, a single specific HLA-restricted epitope or multiple specific HL A-restricted epitopes of HPIV3.

In some embodiments, HPIV3 specific T-cell subpopulations can be generated as described below using one or more antigenic peptides to HPIV3. In some embodiments, the HPIV3-specific T-cell subpopulations are generated using one or more antigenic peptides to HPIV3, or a modified or heteroclitic peptide derived from a HPIV3 antigenic peptide. In some embodiments, HPIV3-specific T-cell subpopulations are generated using a HPIV3 antigen library comprising a pool of peptides (for example, amino acid sequences that are from about 10 to about 20 amino acids from one or a combination of sequence identifiers disclosed herein, such as 15-mers containing amino acid overlap (for example 11 amino acids of overlap) between each sequence formed by scanning the protein having the amino acid sequence of SEQ ID NO: 1 (amino acid sequence of HN, strain Wash/47885157).

In some embodiments, HPIV3-specific T-cell subpopulations are generated using a HPIV3 antigen library comprising a pool of peptides (for example 15mers) containing amino acid overlap (for example 11 amino acids of overlap) between each sequence formed by scanning the protein having the amino acid sequence of SEQ ID NO: 2 (amino acid sequence of Fus, strain Wash/47885/57).

In some embodiments, HPIV3-specific T-cell subpopulations are generated using a HPIV3 antigen library comprising a pool of peptides (for example 15mers) containing amino acid overlap (for example 11 amino acids of overlap) between each sequence formed by scanning the protein having the amino acid sequence of SEQ ID NO: 3 (amino acid sequence of Mat, strain Wash/47885/57).

In some embodiments, HPIV3-specific T-cell subpopulations are generated using a HPIV3 antigen library comprising a pool of peptides (for example 15mers) containing amino acid overlap (for example 11 amino acids of overlap) between each sequence formed by scanning the protein having the amino acid sequence of SEQ ID NO: 4 (amino acid sequence of NP, strain Wash/47885/57).

In some embodiments, HPIV3-specific T-cell subpopulations are generated using a HPIV3 antigen library comprising a pool of peptides (for example 15mers) containing amino acid overlap (for example 11 amino acids of overlap) between each sequence formed by scanning the protein having the amino acid sequence of SEQ ID NO: 5 (amino acid sequence of P, strain Wash/47885/57).

In some embodiments, HPIV3-specific T-cell subpopulations are generated using a HPIV3 antigen library comprising a pool of peptides (for example 15mers) containing amino acid overlap (for example 11 amino acids of overlap) between each sequence formed by scanning the protein having the amino acid sequence of SEQ ID NO: 6 (amino acid sequence of L, strain Wash/47885/57).

The antigenic peptide library can be obtained commercially, for example, by custom ordering from A&A Labs (San Diego, Calif.). In some embodiments, the HPIV3-specific T-cell subpopulations are generated using an overlapping antigenic library made up of the entire HPIV3 polyprotein. In some embodiments, the HPIV3-specific T-cell subpopulations are generated using an overlapping antigenic library made up of less than the entire HPIV3 polyprotein.

In some embodiments, the HPIV3-specific T-cell subpopulations are generated using one or more antigenic peptides of a HPIV3 polyprotein, or a modified or heteroclitic peptide derived from a HPIV3 peptide. In some embodiments, the HPIV3-specific T-cell subpopulations are generated with peptides that recognize class I MHC molecules. In some embodiments, the HPIV3-specific T-cell subpopulations are generated with peptides that recognize class II MHC molecules. In some embodiments, the HPIV3-specific T-cell subpopulations are generated with peptides that recognize both class I and class II MHC molecules.

In some embodiments, the T-cell subpopulation is primed with a peptide mix of a single HPIV3 antigen, wherein the peptide mix comprises antigenic epitopes derived from the single HPIV3 antigen based on one or more of the donor's HLA phenotypes, for example, the peptides are restricted through one or more of the cell donor's HLA alleles such as, but not limited to, HLA-A, HLA-B, and HLA-DR. By including specifically selected donor HLA-restricted peptides from the HPIV3 antigen in the peptide mix for priming and expanding each T-cell subpopulation, a T-cell subpopulation can be generated that provides greater HPIV3 targeted activity through one or more of a donor's HLA-A alleles, HLA-B alleles, or HLA-DR alleles, or a combination thereof, improving potential efficacy of the T-cell subpopulation for patients that share at least one HLA allele with the donor. In addition, by generating a T-cell subpopulation with HPIV3-specific antigen targeted activity through more than one donor HLA allele, a single donor T-cell subpopulation may be included in a disclosed composition for multiple recipients with different HLA profiles by matching one or more donor HLA alleles showing HPIV3 activity. In some embodiments therefore, each T-cell in the disclosed T-cell subpopulation comprises one or more T-cell receptors (TCRs) that recognize one or more epitopes of the HPIV3 antigen and reactive to one or more HLA alleles. In some embodiments, the HLA-A alleles are selected from a group comprising HLA-A*01, HLA-A*02:01, HLA-A*03, HLA-A*11:01, HLA-A*24:02, HLA-A*26, and HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35.01, and HLA-B*58:02 In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, and HLA-DRB1*1501 (DR2b). In some embodiments, the master mix of peptides includes both an overlapping peptide library and specifically selected HLA-restricted peptides generated by determining the HLA profile of the donor source.

In some embodiments, the HPIV3 peptides used to prime and expand a T-cell subpopulation includes specifically selected HLA-restricted peptides generated by determining the HLA profile of the donor source, and including peptides derived from HPIV3 that best match the donor's HLA. In some embodiments, the HPIV3 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, HG., Bachmann, J., Emmerich, N. et al., SYFPEITHI: database for MHC ligands and peptide motifs. Immunogenetics (1999) 50: 213 (available at doi.org/10.1007/s002510050595, incorporated by reference).

As provided herein, the HLA profile of a donor cell source can be determined, and T-cell subpopulations targeting HPIV3 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides. For example, if the donor cell source has an HLA profile that is HLA-A*01/*02:01; HLA-B*15:01/*18; and HLA-DRB1*0101/*0301, then the HPIV3 peptides used to prime and expand the HPIV3-specific T-cell subpopulation are restricted to the specific HLA profile, and may include the peptides for HLA-A*01, the peptides for HLA-A*02:01, the peptides for HLA-B*15:01/*18, and the peptides for HLA-DRB1*0101/*0301. In some embodiments, the mastermix of peptides includes both an overlapping peptide library and specifically selected HLA-restricted peptides generated by determining the HLA profile of the donor source.

In some embodiments, the HPIV3-specific T-cell subpopulation is primed and expanded with one or more Mat-derived peptides selected from Table 1 (SEQ ID NO: 7-19). In some embodiments, the HPIV3-specific T-cell subpopulation is primed and expanded with Mat-derived peptides selected from Table 1 (SEQ ID NO: 7-19). In some embodiments, the HPIV3-specific T-cell subpopulation is primed and expanded with Mat-derived peptides comprising the peptides of Table 1 (SEQ ID NO: 7-19). In some embodiments, the HPIV3-specific T-cell subpopulation is primed and expanded with Mat-derived peptides comprising the peptides of Table 1 (SEQ ID NO: 7-19) and at least one additional set of peptides based on the donor cell source HLA profile.

T-Cell Compositions. The T-cell composition according to the present disclosure can comprise one or a plurality of T-cell subpopulations and each T-cell subpopulation is specific for one or a plurality of HPIV3 antigens, provided that at least one T-cell subpopulation is specific for a single HPIV3 antigen. In some embodiments, the disclosed composition comprises only one T-cell subpopulation specific for a single HPIV3 antigen. In some embodiments, the disclosed composition comprises a first T-cell subpopulation and a second T-cell subpopulation, wherein at least one of the first and second T-cell subpopulations is specific for a single HPIV3 antigen. In some embodiments, the disclosed composition comprises a first T-cell subpopulation and a second T-cell subpopulation, wherein each T-cell subpopulation is specific for a different HPIV3 antigen. In some embodiments, the disclosed composition comprises a first T-cell subpopulation, a second T-cell subpopulation, and a third T-cell subpopulation, wherein at least one of the first, second and third T-cell subpopulations is specific for a single HPIV3 antigen. In some embodiments, the disclosed composition comprises a first T-cell subpopulation, a second T-cell subpopulation, and a third T-cell subpopulation, wherein each T-cell subpopulation is specific for a different HPIV3 antigen. In some embodiments, the disclosed composition comprises a first T-cell subpopulation, a second T-cell subpopulation, a third T-cell subpopulation, and a fourth T-cell subpopulation, wherein at least one of the first, second, third and fourth T-cell subpopulations is specific for a single HPIV3 antigen. In some embodiments, the disclosed composition comprises a first T-cell subpopulation, a second T-cell subpopulation, a third T-cell subpopulation, and a fourth T-cell subpopulation, wherein each T-cell subpopulation is specific for a different HPIV3 antigen. In some embodiments, the disclosed composition comprises up to about five T-cell subpopulations and at least one of the T-cell subpopulations is specific for a single HPIV3 antigen. In some embodiments, the disclosed composition comprises up to about five T-cell subpopulations and each T-cell subpopulation is specific for a different HPIV3 antigen. In some embodiments, the disclosure relates to a library of T-cell subpopulations specific for one or a plurality of HPIV3 antigens, such library being stored frozen for no less than about 1, 10, 15, 30, 45, 90, 120, 150, 180 or more days.

The ratio of the T-cell subpopulations in the disclosed composition may be selected based on the knowledge of the patient's viral load or the healthcare provider's best judgment. In some embodiments, each of the T-cell subpopulations in the disclosed composition is in a defined ratio based on either total cell number or normalized cell activity. In some embodiments, each of the T-cell subpopulations in the disclosed composition is in about an equal ratio. In some embodiments, the ratio or percentage of each T-cell subpopulation is normalized based on the measured activity of each T-cell subpopulation against the HPIV3 as measured by, for example, but not limited to, the EliSpot assay.

In some embodiments, the disclosed composition comprises a first T-cell subpopulation and a second T-cell subpopulation, wherein the first and second T-cell subpopulations are in about an equal ratio based on either total cell number or normalized cell activity. In some embodiments, the disclosed composition comprises a first T-cell subpopulation and a second T-cell subpopulation, wherein the percentage of the first subpopulation in the disclosed composition is from about 40% to about 60% of the disclosed composition based on either total cell number or normalized cell activity. In some embodiments, the disclosed composition comprises three T-cell subpopulations, wherein the disclosed composition comprises (i) at least about 45%, 50%, 55%, 60%, 65%, 70%, 75%, 80%, or 85% of a first T-cell subpopulation, (ii) at least about 5%, 10%, 15%, 20%, or 25% of a second T-cell subpopulation, and (iii) at least about 10%, 15%, 20%, 25%, 30%, or 35% of a third T-cell subpopulation, wherein the percentage adds to 100% based on either total cell number or normalized cell activity. The ratios of the T-cell subpopulations in the disclosed composition may be selected based on the knowledge of the patient's infecting HPIV3 or the healthcare provider's best judgement. In some embodiments, the disclosed composition comprises four T-cell subpopulations, wherein the disclosed composition comprises (i) at least about 45%, 50%, 55%, 60%, 65%, 70%, 75%, 80%, or 85% of a first T-cell subpopulation, (ii) at least about 5%, 10%, 15%, 20%, 25%, 30%, 35%, 40,% or 45% of a second T-cell subpopulation, (iii) at least about 5%, 10%, 15%, 20%, 25%, 30%, 35%, 40% or 45% of a third T-cell subpopulation, and (iv) at least about 5%, 10%, 15%, 20%, 25%, 30%, 35%, 40% or 45% of a fourth T-cell subpopulation, wherein the percentage adds to 100% based on either total cell number or normalized cell activity. In some embodiments, the disclosed composition comprises five T-cell subpopulations, wherein the disclosed composition comprises (i) at least about 45%, 50%, 55%, 60/o, 65%, 70%, 75%, 80% of a first T-cell subpopulation, (ii) at least about 5%, 10%, 15%, 20%, 25%, 30%, 35%, or 40% of a second T-cell subpopulation, (iii) at least about 5%, 10%, 15%, 20%, 25%, 30%, 35%, or 40% of a third T-cell subpopulation, (iv) at least about 5%, 10%, 15%, 20%, 25%, 30%, 35%, or 40% of a fourth T-cell subpopulation, and (v) at least about 5%, 10%, 15%, 20%, 25%, 30%, 35%, or 40% of a fifth T-cell subpopulation, wherein the percentage adds to 100% based on either total cell number or normalized cell activity.

In some embodiments, the ratio or percentage of each T-cell subpopulation is normalized based on the measured activity of each T-cell subpopulation against the HPIV3 antigen as measured by, for example, but not limited to, the ELISpot assay. In some embodiments, the percentage of the T-cell subpopulations is based on the HPIV3 antigen profile of the HPIV3 strain found in the patient such that the percentage of each T-cell subpopulation correlates with the HPIV3 antigen profile of the HPIV3 strain found in the patient. In some embodiments, each of the T-cell subpopulations is specific to one or a combination of: HN, Fus, Mat, NP, P and L, or a functional fragment thereof.

In some embodiments, the disclosed composition comprises only one T-cell subpopulation, wherein the T-cell subpopulation is specific for a single HPIV3 antigen selected from HN, Fus, Mat, NP, P and L, or a functional fragment thereof. In some embodiments, the disclosed composition comprises only one T-cell subpopulation that is specific to HN or a functional fragment thereof. In some embodiments, the disclosed composition comprises only one T-cell subpopulation that is specific to Fus or a functional fragment thereof. In some embodiments, the disclosed composition comprises only one T-cell subpopulation that is specific to Mat or a functional fragment thereof. In some embodiments, the disclosed composition comprises only one T-cell subpopulation that is specific to NP or a functional fragment thereof. In some embodiments, the disclosed composition comprises only one T-cell subpopulation that is specific to P or a functional fragment thereof. In some embodiments, the disclosed composition comprises only one T-cell subpopulation that is specific to L or a functional fragment thereof.

In some embodiments, the disclosed composition comprises at least two T-cell subpopulations, wherein at least one T-cell subpopulation is specific for a single HPIV3 antigen selected from HN, Fus, Mat, NP, P and L, or a functional fragment thereof. In some embodiments, the disclosed composition comprises at least two T-cell subpopulations, wherein at least one T-cell subpopulation is specific to HN or a functional fragment thereof. In some embodiments, the disclosed composition comprises at least two T-cell subpopulations, wherein at least one T-cell subpopulation is specific to Fus or a functional fragment thereof. In some embodiments, the disclosed composition comprises at least two T-cell subpopulations, wherein at least one T-cell subpopulation is specific to Mat or a functional fragment thereof. In some embodiments, the disclosed composition comprises at least two T-cell subpopulations, wherein at least one T-cell subpopulation is specific to NP or a functional fragment thereof. In some embodiments, the disclosed composition comprises at least two T-cell subpopulations, wherein at least one T-cell subpopulation is specific to P or a functional fragment thereof. In some embodiments, the disclosed composition comprises at least two T-cell subpopulations, wherein at least one T-cell subpopulation is specific to L or a functional fragment thereof.

In some embodiments, the disclosed composition comprises at least two T-cell subpopulations, each T-cell subpopulation is specific for a single HPIV3 antigen selected from HN, Fus, Mat, NP, P and L, or a functional fragment thereof. In some embodiments, the disclosed composition comprises at least two T-cell subpopulations, wherein the first T-cell subpopulation is specific to HN or a functional fragment thereof, and the second T-cell subpopulation is specific to any one of HN, Fus, Mat, NP, P and L, or a functional fragment thereof. In some embodiments, the disclosed composition comprises at least two T-cell subpopulations, wherein the first T-cell subpopulation is specific to Fus or a functional fragment thereof, and the second T-cell subpopulation is specific to any one of HN, Fus, Mat, NP, P and L, or a functional fragment thereof. In some embodiments, the disclosed composition comprises at least two T-cell subpopulations, wherein the first T-cell subpopulation is specific to Mat or a functional fragment thereof, and the second T-cell subpopulation is specific to any one of HN, Fus, Mat, NP, P and L, or a functional fragment thereof. In some embodiments, the disclosed composition comprises at least two T-cell subpopulations, wherein the first T-cell subpopulation is specific to NP or a functional fragment thereof, and the second T-cell subpopulation is specific to any one of HN, Fus, Mat, NP, P and L, or a functional fragment thereof. In some embodiments, the disclosed composition comprises at least two T-cell subpopulations, wherein the first T-cell subpopulation is specific to P or a functional fragment thereof, and the second T-cell subpopulation is specific to any one of HN, Fus, Mat, NP, P and L, or a functional fragment thereof. In some embodiments, the disclosed composition comprises at least two T-cell subpopulations, wherein the first T-cell subpopulation is specific to L or a functional fragment thereof, and the second T-cell subpopulation is specific to any one of HN, Fus, Mat, NP, P and L, or a functional fragment thereof.

In some embodiments, the disclosed composition comprises at least two T-cell subpopulations, each T-cell subpopulation is specific for a different HPIV3 antigen selected from HN, Fus, Mat, NP, P and L, or a functional fragment thereof. In some embodiments, the disclosed composition comprises at least two T-cell subpopulations, wherein the first T-cell subpopulation is specific to HN or a functional fragment thereof, and the second T-cell subpopulation is specific to any one of Fus, Mat, NP, P and L, or a functional fragment thereof. In some embodiments, the disclosed composition comprises at least two T-cell subpopulations, wherein the first T-cell subpopulation is specific to Fus or a functional fragment thereof, and the second T-cell subpopulation is specific to any one of Mat, NP, P and L, or a functional fragment thereof. In some embodiments, the disclosed composition comprises at least two T-cell subpopulations, wherein the first T-cell subpopulation is specific to Mat or a functional fragment thereof, and the second T-cell subpopulation is specific to any one of NP, P and L, or a functional fragment thereof. In some embodiments, the disclosed composition comprises at least two T-cell subpopulations, wherein the first T-cell subpopulation is specific to NP or a functional fragment thereof, and the second T-cell subpopulation is specific to any one of P and L, or a functional fragment thereof. In some embodiments, the disclosed composition comprises at least two T-cell subpopulations, wherein the first T-cell subpopulation is specific to P or a functional fragment thereof, and the second T-cell subpopulation is specific to L, or a functional fragment thereof.

In some embodiments, the disclosed composition comprises at least three T-cell subpopulations, wherein at least one T-cell subpopulation is specific for a single HPIV3 antigen selected from HN, Fus, Mat, NP, P and L, or a functional fragment thereof. In some embodiments, the disclosed composition comprises at least three T-cell subpopulations, wherein at least one T-cell subpopulation is specific to HN or a functional fragment thereof. In some embodiments, the disclosed composition comprises at least three T-cell subpopulations, wherein at least one T-cell subpopulation is specific to Fus or a functional fragment thereof. In some embodiments, the disclosed composition comprises at least three T-cell subpopulations, wherein at least one T-cell subpopulation is specific to Mat or a functional fragment thereof. In some embodiments, the disclosed composition comprises at least three T-cell subpopulations, wherein at least one T-cell subpopulation is specific to NP or a functional fragment thereof. In some embodiments, the disclosed composition comprises at least three T-cell subpopulations, wherein at least one T-cell subpopulation is specific to P or a functional fragment thereof. In some embodiments, the disclosed composition comprises at least three T-cell subpopulations, wherein at least one T-cell subpopulation is specific to L or a functional fragment thereof.

In some embodiments, the disclosed composition comprises at least three T-cell subpopulations, each T-cell subpopulation is specific for a single HPIV3 antigen selected from HN, Fus, Mat, NP, P and L, or a functional fragment thereof. In some embodiments, the disclosed composition comprises at least three T-cell subpopulations, each T-cell subpopulation is specific for a different HPIV3 antigen selected from HN, Fus, Mat, NP, P and L, or a functional fragment thereof. In some embodiments, the disclosed composition comprises at least three T-cell subpopulations, wherein the first T-cell subpopulation is specific to HN or a functional fragment thereof, the second T-cell subpopulation is specific to Fus or a functional fragment thereof, and the third T-cell subpopulation is specific to any of Mat, NP, P and L, or a functional fragment thereof. In some embodiments, the disclosed composition comprises at least three T-cell subpopulations, wherein the first T-cell subpopulation is specific to HN or a functional fragment thereof, the second T-cell subpopulation is specific to Mat or a functional fragment thereof, and the third T-cell subpopulation is specific to any of NP, P and L, or a functional fragment thereof. In some embodiments, the disclosed composition comprises at least three T-cell subpopulations, wherein the first T-cell subpopulation is specific to HN or a functional fragment thereof, the second T-cell subpopulation is specific to NP or a functional fragment thereof, and the third T-cell subpopulation is specific to any of P and L, or a functional fragment thereof. In some embodiments, the disclosed composition comprises at least three T-cell subpopulations, wherein the first T-cell subpopulation is specific to HN or a functional fragment thereof, the second T-cell subpopulation is specific to P or a functional fragment thereof, and the third T-cell subpopulation is specific to L, or a functional fragment thereof. In some embodiments, the disclosed composition comprises at least three T-cell subpopulations, wherein the first T-cell subpopulation is specific to Fus or a functional fragment thereof, the second T-cell subpopulation is specific to Mat or a functional fragment thereof, and the third T-cell subpopulation is specific to any of NP, P and L, or a functional fragment thereof. In some embodiments, the disclosed composition comprises at least three T-cell subpopulations, wherein the first T-cell subpopulation is specific to Fus or a functional fragment thereof, the second T-cell subpopulation is specific to NP or a functional fragment thereof, and the third T-cell subpopulation is specific to any of P and L, or a functional fragment thereof. In some embodiments, the disclosed composition comprises at least three T-cell subpopulations, wherein the first T-cell subpopulation is specific to Fus or a functional fragment thereof, the second T-cell subpopulation is specific to P or a functional fragment thereof, and the third T-cell subpopulation is specific to L, or a functional fragment thereof. In some embodiments, the disclosed composition comprises at least three T-cell subpopulations, wherein the first T-cell subpopulation is specific to Mat or a functional fragment thereof, the second T-cell subpopulation is specific to NP or a functional fragment thereof, and the third T-cell subpopulation is specific to any of P and L, or a functional fragment thereof. In some embodiments, the disclosed composition comprises at least three T-cell subpopulations, wherein the first T-cell subpopulation is specific to Mat or a functional fragment thereof, the second T-cell subpopulation is specific to P or a functional fragment thereof, and the third T-cell subpopulation is specific to L, or a functional fragment thereof. In some embodiments, the disclosed composition comprises at least three T-cell subpopulations, wherein the first T-cell subpopulation is specific to NP or a functional fragment thereof, the second T-cell subpopulation is specific to P or a functional fragment thereof, and the third T-cell subpopulation is specific to L, or a functional fragment thereof.

In some embodiments, the disclosed composition comprises at least four T-cell subpopulations, wherein at least one I-cell subpopulation is specific for a single HPIV3 antigen selected from HN, Fus, Mat, NP, P and L, or a functional fragment thereof. In some embodiments, the disclosed composition comprises at least four T-cell subpopulations, wherein at least one T-cell subpopulation is specific to HN or a functional fragment thereof. In some embodiments, the disclosed composition comprises at least four T-cell subpopulations, wherein at least one T-cell subpopulation is specific to Fus or a functional fragment thereof. In some embodiments, the disclosed composition comprises at least four T-cell subpopulations, wherein at least one T-cell subpopulation is specific to Mat or a functional fragment thereof. In some embodiments, the disclosed composition comprises at least four T-cell subpopulations, wherein at least one T-cell subpopulation is specific to NP or a functional fragment thereof. In some embodiments, the disclosed composition comprises at least four T-cell subpopulations, wherein at least one T-cell subpopulation is specific to P or a functional fragment thereof. In some embodiments, the disclosed composition comprises at least four T-cell subpopulations, wherein at least one T-cell subpopulation is specific to L or a functional fragment thereof.

In some embodiments, the disclosed composition comprises at least four T-cell subpopulations, each T-cell subpopulation is specific for a single HPIV3 antigen selected from HN, Fus, Mat, NP, P and L, or a functional fragment thereof. In some embodiments, the disclosed composition comprises at least four T-cell subpopulations, each T-cell subpopulation is specific for a different HPIV3 antigen selected from HN, Fus, Mat, NP, P and L, or a functional fragment. In some embodiments, the disclosed composition comprises at least four T-cell subpopulations, wherein the first T-cell subpopulation is specific to HN or a functional fragment thereof, the second T-cell subpopulation is specific to Fus or a functional fragment thereof, the third T-cell subpopulation is specific to Mat, or a functional fragment thereof, and the fourth T-cell subpopulation is specific to any of NP, P or L, or a functional fragment. In some embodiments, the disclosed composition comprises at least four T-cell subpopulations, wherein the first T-cell subpopulation is specific to HN or a functional fragment thereof, the second T-cell subpopulation is specific to Fus or a functional fragment thereof, the third T-cell subpopulation is specific to NP, or a functional fragment thereof, and the fourth T-cell subpopulation is specific to any of P or L, or a functional fragment. In some embodiments, the disclosed composition comprises at least four T-cell subpopulations, wherein the first T-cell subpopulation is specific to HN or a functional fragment thereof, the second T-cell subpopulation is specific to Fus or a functional fragment thereof, the third T-cell subpopulation is specific to P, or a functional fragment thereof, and the fourth T-cell subpopulation is specific to L, or a functional fragment. In some embodiments, the disclosed composition comprises at least four T-cell subpopulations, wherein the first T-cell subpopulation is specific to HN or a functional fragment thereof, the second T-cell subpopulation is specific to Mat or a functional fragment thereof, the third T-cell subpopulation is specific to NP, or a functional fragment thereof, and the fourth T-cell subpopulation is specific to any of P or L, or a functional fragment. In some embodiments, the disclosed composition comprises at least four T-cell subpopulations, wherein the first T-cell subpopulation is specific to HN or a functional fragment thereof, the second T-cell subpopulation is specific to Mat or a functional fragment thereof, the third T-cell subpopulation is specific to P, or a functional fragment thereof, and the fourth T-cell subpopulation is specific to L, or a functional fragment. In some embodiments, the disclosed composition comprises at least four T-cell subpopulations, wherein the first T-cell subpopulation is specific to HN or a functional fragment thereof, the second T-cell subpopulation is specific to NP or a functional fragment thereof, the third T-cell subpopulation is specific to P, or a functional fragment thereof, and the fourth T-cell subpopulation is specific to L, or a functional fragment. In some embodiments, the disclosed composition comprises at least four T-cell subpopulations, wherein the first T-cell subpopulation is specific to Fus or a functional fragment thereof, the second T-cell subpopulation is specific to Mat or a functional fragment thereof, the third T-cell subpopulation is specific to NP, or a functional fragment thereof, and the fourth T-cell subpopulation is specific to any of P or L, or a functional fragment. In some embodiments, the disclosed composition comprises at least four T-cell subpopulations, wherein the first T-cell subpopulation is specific to Fus or a functional fragment thereof, the second T-cell subpopulation is specific to Mat or a functional fragment thereof, the third T-cell subpopulation is specific to P, or a functional fragment thereof, and the fourth T-cell subpopulation is specific to L, or a functional fragment. In some embodiments, the disclosed composition comprises at least four T-cell subpopulations, wherein the first T-cell subpopulation is specific to Fus or a functional fragment thereof, the second T-cell subpopulation is specific to NP or a functional fragment thereof, the third T-cell subpopulation is specific to P, or a functional fragment thereof, and the fourth T-cell subpopulation is specific to L, or a functional fragment. In some embodiments, the disclosed composition comprises at least four T-cell subpopulations, wherein the first T-cell subpopulation is specific to Mat or a functional fragment thereof, the second T-cell subpopulation is specific to NP or a functional fragment thereof, the third T-cell subpopulation is specific to P, or a functional fragment thereof, and the fourth T-cell subpopulation is specific to L, or a functional fragment.

In some embodiments, the disclosed composition comprises at least five T-cell subpopulations, wherein at least one T-cell subpopulation is specific for a single HPIV3 antigen selected from HN, Fus, Mat, NP, P and L, or a functional fragment thereof. In some embodiments, the disclosed composition comprises at least five T-cell subpopulations, wherein at least one T-cell subpopulation is specific to HN or a functional fragment thereof. In some embodiments, the disclosed composition comprises at least five T-cell subpopulations, wherein at least one T-cell subpopulation is specific to Fus or a functional fragment thereof. In some embodiments, the disclosed composition comprises at least five T-cell subpopulations, wherein at least one T-cell subpopulation is specific to Mat or a functional fragment thereof. In some embodiments, the disclosed composition comprises at least five T-cell subpopulations, wherein at least one T-cell subpopulation is specific to NP or a functional fragment thereof. In some embodiments, the disclosed composition comprises at least five T-cell subpopulations, wherein at least one T-cell subpopulation is specific to P or a functional fragment thereof. In some embodiments, the disclosed composition comprises at least five T-cell subpopulations, wherein at least one T-cell subpopulation is specific to L or a functional fragment thereof.

In some embodiments, the disclosed composition comprises at least five T-cell subpopulations, each T-cell subpopulation is specific for a single HPIV3 antigen selected from HN, Fus, Mat, NP, P and L, or a functional fragment thereof. In some embodiments, the disclosed composition comprises at least five T-cell subpopulations, each T-cell subpopulation is specific for a different HPIV3 antigen selected from HN, Fus, Mat, NP, P and L, or a functional fragment. In some embodiments, the disclosed composition comprises at least five T-cell subpopulations, wherein the first T-cell subpopulation is specific to HN or a functional fragment thereof, the second T-cell subpopulation is specific to Fus or a functional fragment thereof, the third T-cell subpopulation is specific to Mat, or a functional fragment thereof, the fourth T-cell subpopulation is specific to NP, or a functional fragment thereof, and the fifth T-cell subpopulation is specific to any of P or L, or a functional fragment. In some embodiments, the disclosed composition comprises at least five T-cell subpopulations, wherein the first T-cell subpopulation is specific to HN or a functional fragment thereof, the second T-cell subpopulation is specific to Fus or a functional fragment thereof, the third T-cell subpopulation is specific to Mat, or a functional fragment thereof, the fourth T-cell subpopulation is specific to P, or a functional fragment thereof, and the fifth T-cell subpopulation is specific to L, or a functional fragment. In some embodiments, the disclosed composition comprises at least five T-cell subpopulations, wherein the first T-cell subpopulation is specific to HN or a functional fragment thereof, the second T-cell subpopulation is specific to Fus or a functional fragment thereof, the third T-cell subpopulation is specific to NP, or a functional fragment thereof, the fourth T-cell subpopulation is specific to P, or a functional fragment thereof, and the fifth T-cell subpopulation is specific to L, or a functional fragment. In some embodiments, the disclosed composition comprises at least five T-cell subpopulations, wherein the first T-cell subpopulation is specific to HN or a functional fragment thereof, the second T-cell subpopulation is specific to Mat or a functional fragment thereof, the third T-cell subpopulation is specific to NP, or a functional fragment thereof, the fourth T-cell subpopulation is specific to P, or a functional fragment thereof, and the fifth T-cell subpopulation is specific to L, or a functional fragment. In some embodiments, the disclosed composition comprises at least five T-cell subpopulations, wherein the first T-cell subpopulation is specific to Fus or a functional fragment thereof, the second T-cell subpopulation is specific to Mat or a functional fragment thereof, the third T-cell subpopulation is specific to NP, or a functional fragment thereof, the fourth T-cell subpopulation is specific to P, or a functional fragment thereof, and the fifth T-cell subpopulation is specific to L, or a functional fragment.

In some embodiments, the disclosed composition comprises at least one T-cell subpopulation that is specific for a single HPIV3 antigen comprising at least one epitope comprising at least 75, 85, 90, 95, 99 or <100% sequence identity to SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19. In some embodiments, the disclosed composition comprises at least one T-cell subpopulation that is specific for a single HPIV3 antigen comprising at least one epitope comprising at least 85% sequence identity to SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19. In some embodiments, the disclosed composition comprises at least one T-cell subpopulation that is specific for a single HPIV3 antigen comprising at least one epitope comprising at least 90% sequence identity to SEQ ID NO: 7, SEQ ID NO. 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19. In some embodiments, the disclosed composition comprises at least one T-cell subpopulation that is specific for a single HPIV3 antigen comprising at least one epitope comprising at least 95% sequence identity to SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19. In some embodiments, the disclosed composition comprises at least one T-cell subpopulation that is specific for a single HPIV3 antigen comprising at least one epitope comprising at least 96% sequence identity to SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19. In some embodiments, the disclosed composition comprises at least one T-cell subpopulation that is specific for a single HPIV3 antigen comprising at least one epitope comprising at least 97% sequence identity to SEQ ID NO: 7, SEQ ID NO: 8, SEQ ID NO: 9, SEQ ID NO: 10, SEQ ID NO: 11, SEQ ID NO. 12, SEQ ID NO. 13, SEQ ID NO: 14, SEQ ID NO. 15, SEQ ID NO: 16, SEQ ID NO: 17, SEQ ID NO: 18 or SEQ ID NO: 19. In some embodiments, the disclosed composition comprises at least one T-cell subpopulation that is specific for a single HPIV3 antigen comprising at least one epitope comprising at least 98% sequence identity to SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19. In some embodiments, the disclosed composition comprises at least one T-cell subpopulation that is specific for a single HPIV3 antigen comprising at least one epitope comprising at least 99% sequence identity to SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19. In some embodiments, the disclosed composition comprises at least one T-cell subpopulation that is specific for a single HPIV3 antigen comprising at least one epitope comprising the amino acid sequence of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19.

In some embodiments, the mononuclear cell sample from which the T-cell subpopulations are isolated is originated from the same human subject to which the composition is also administered (autologous). In some embodiments, the mononuclear cell sample from which the T-cell subpopulations are isolated is originated from a cell donor (allogeneic). In certain embodiments, the allogeneic T-cell subpopulation composition has at least one HLA allele or HLA allele combination in common with the patient. In certain embodiments, the allogeneic T-cell subpopulation composition has more than one HLA allele or HLA allele combination in common with the patient. In certain embodiments, the HPIV3 antigen activity of the disclosed composition is through at least one HLA allele or HLA allele combination in common with the patient. In certain embodiments, the allogeneic T-cell subpopulations comprising the disclosed composition are recognized through the same shared HLA restriction. In certain embodiments, the allogeneic T-cell subpopulations comprising the disclosed composition are recognized through different shared HLA restrictions.

In another aspect, the present disclosure provides a method of treating a disease or disorder comprising administering a therapeutically effective amount of the T-cell composition disclosed herein to a patient, typically a human, in need thereof.

In some embodiments, the method further comprises isolating a mononuclear cell sample from the patient, typically a human to which the disclosed composition is administered (autologous), wherein the disclosed composition comprises T-cell subpopulations made from the mononuclear cell sample.

In some embodiments, the method further comprises isolating a mononuclear cell sample from a cell donor (allogeneic), wherein the disclosed composition comprises T-cell subpopulations made from the mononuclear cell sample. In certain embodiments, the allogeneic disclosed composition has at least one HLA allele or HLA allele combination in common with the patient. In certain embodiments, the allogeneic disclosed composition has more than one HLA allele or HLA allele combination in common with the patient. In certain embodiments, the HPIV3 antigen activity of the disclosed composition is through at least one HLA allele or HLA allele combination in common with the patient. In certain embodiments, the HPIV3 antigen activity of the disclosed composition is through more than one HLA allele or HLA allele combination in common with the patient. In certain embodiments, the allogeneic T-cell subpopulations comprising the disclosed composition are recognized through the same shared HLA restriction. In certain embodiments, the allogeneic T-cell subpopulations comprising the disclosed composition are recognized through different shared HLA restrictions. In certain embodiments, the disclosed composition selected has the most shared HLA alleles or allele combinations and the highest HPIV3 antigen specificity.

In certain embodiments, the method further comprises selecting the disclosed composition based on the levels of circulating HPIV3-specific T-cells present in the patient after administration of a disclosed composition. Methods of measuring the levels of circulating HPIV3-specific T-cells present in the patient are known in the art and non-limiting exemplary methods include Elispot assay, TCR sequencing, intracellular cytokine staining, and through the uses of MHC-peptide multimers.

Method of Treating a Patient by Administering a Disclosed Composition. In another aspect, the present disclosure provides a method of treating a subject having HPIV3 infection by administering one of the disclosed compositions. In some embodiments, the patient is infected by HPIV3 and at least one of cytomegalovirus (CMV), Epstein-Barr virus (EBV), Adenovirus, human herpesvirus 6 (HHV-6). In some embodiments, the patient is infected by HPIV3. In some embodiments, the present disclosure includes a method to treat a patient with a HPIV3 infection, typically a human, by administering a therapeutically effective amount of a disclosed composition described herein. In some embodiments, the patient being treated by the method of the present disclosure suffers acute respiratory infections and/or is immunodeficiency due to genetic disorders, solid organ transplantation, or hematopoietic stem cell transplant (HSCT).

Human Parainfluenza Virus-1 (HPIV1) contains viral proteins that share a high degree of sequence identity with HPIV3. It has been observed that T-cells generated to target HPIV3 also have cross-reactivity with the corresponding viral proteins in HPIV1. It is therefore foreseeable that HPIV1 infections may be treated using T-cell subpopulations expanded against HPIV3 antigens that display cross-reactivity. Using epitope mapping, this may also be feasible using banked, partially-HLA matched HPIV3-specific T-cell subpopulations. Accordingly, in another aspect, the present disclosure provides a method of treating a subject having HPIV1 infection by administering one of the disclosed compositions. In some embodiments, the present disclosure includes a method to treat a patient with a HPIV1 infection, typically a human, by administering a therapeutically effective amount of a disclosed composition described herein. In some embodiments, the disclosed composition used for treating HPIV1 infection comprises at least one T-cell subpopulation that is specific for a single HPIV3 antigen and cross-reacts with at least one HPIV1 antigen. In some embodiments, the method of treating a subject having HPIV1 infection provided in the present disclosure comprises identifying one or a plurality of HPIV1 antigens associated with a tissue or cell of the subject for targeting and selecting one or a plurality of banked HPIV3-specific T-cell subpopulations based on sequence homology between the one or plurality of HPIV1 antigens and the HPIV3 antigens used for generating the banked HPIV3-specific T-cell subpopulations. In some embodiments, the sequence homology is at least about 40% sequence identity. In some embodiments, the sequence homology is at least about 45% sequence identity. In some embodiments, the sequence homology is at least about 50% sequence identity. In some embodiments, the sequence homology is at least about 55% sequence identity. In some embodiments, the sequence homology is at least about 60% sequence identity. In some embodiments, the sequence homology is at least about 65% sequence identity. In some embodiments, the sequence homology is at least about 70% sequence identity. In some embodiments, the sequence homology is at least about 75% sequence identity. In some embodiments, the sequence homology is at least about 80% sequence identity. In some embodiments, the sequence homology is at least about 85% sequence identity. In some embodiments, the sequence homology is at least about 90% sequence identity. In some embodiments, the sequence homology is at least about 95% sequence identity. In some embodiments, the sequence homology is at least about 96% sequence identity. In some embodiments, the sequence homology is at least about 97% sequence identity. In some embodiments, the sequence homology is at least about 98% sequence identity. In some embodiments, the sequence homology is at least about 99% sequence identity. In some embodiments, the sequence homology is at least about 100% sequence identity.

In some embodiments, the method further comprises determining an HLA subtype of the subject and selecting one or a plurality of banked HPIV3-specific T-cell subpopulations through one or more HLA-alleles shared between the subject and the T-cell subpopulation.

The dose administered may vary. In some embodiments, the disclosed composition is administered to a patient, such as a human, in a dose ranging from about 1×10⁶ cells/m² to 1×10⁷ cells/m² to about 1×10⁸ cells/m². The dose can be a single dose, for example, comprising the combination of all of the T-cell subpopulations comprising the disclosed composition, or multiple separate doses, wherein each dose comprises a separate T-cell subpopulation and the collective separate doses of T-cell subpopulations comprise the total disclosed composition. In some embodiments, the disclosed composition dosage is about 1×10⁶ cells/m², about 2×10⁶ cells/m², about 3×10⁶ cells/m², about 4×10⁶ cells/m², about 5×10⁶ cells/m², about 6×10⁶ cells/m², about 7×10⁶ cells/m², about 8×10⁶ cells/m², about 9×10⁶ cells/m², about 1×10⁷ cells/m², about 2×10⁷ cells/m², about 3×10⁷ cells/m², about 4×10⁷ cells/m², about 5×10⁷ cells/m², about 6×10⁷ cells/m², about 7×10⁷ cells/m², about 8×10⁷ cells/m², about 9×10⁷ cells/m², or about 1×10⁸ cells/m².

The disclosed composition may be administered by any suitable method. In some embodiments, the disclosed composition is administered to a subject, such as a human, as an infusion, and in a particular embodiment, an infusion with a total volume of about 1 to about 10 cc. In some embodiments, the disclosed composition is administered to a subject as 1, 2, 3, 4, 5, 6, 7, 8, 9, or 10 cc infusion. In some embodiments, the disclosed composition when present as an infusion is administered to a subject over about 10, 20, 30, 40, 50, 60 or more minutes to the subject in need thereof.

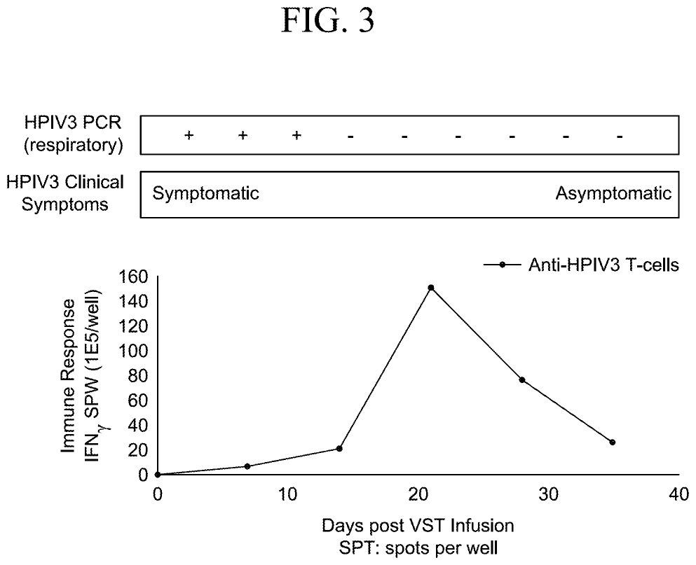

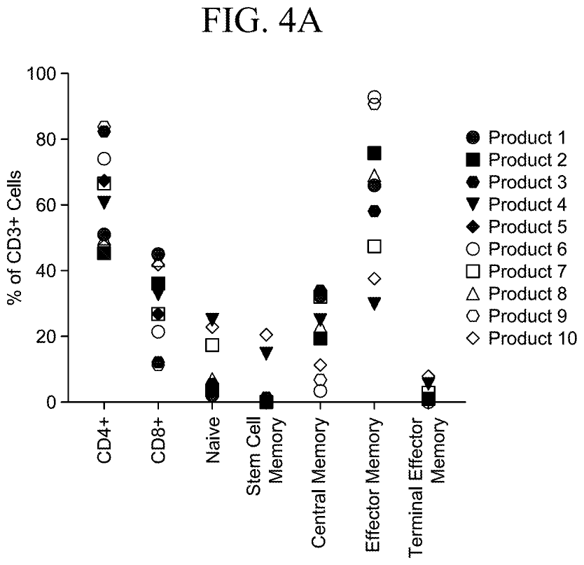

In some embodiments, a subject receiving an infusion has vital signs monitored before, during, and about 1-hour post infusion of the disclosed composition. In certain embodiments, patients with stable disease (SD), partial response (PR), or complete response (CR) up to about 6 weeks after initial infusion may be eligible to receive additional infusions, for example, 1, 2, 3, 4, 5, 6, 7, 8, 9 or 10 additional infusions several weeks apart, for example, up to about 2, 3, 4, 5, 6, 7, 8, 9 or 10 weeks apart. FIG. 2 generally describes confirmation of antiviral activity of T cells and infusion of partially matched VSTs and FIG. 3 describes one example of immune response after VST infusion.

Administration of Disclosed Compostions. Methods for administration of cells for adoptive cell therapy are known and may be used in connection with the provided methods and disclosed compositions. For example, adoptive T cell therapy methods are described, e.g., in US Patent Application Publication No. 2003/0170238 to Gruenberg et al.; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The administration of the disclosed composition may vary. In one aspect, the disclosed composition may be administered to a patient such as a human at an interval selected from about once every 1 week, 2 weeks, 3 weeks, 4 weeks, 5 weeks, 6 weeks, 7 weeks, 8 weeks, or more after the initial administration of the disclosed composition. In a typical embodiment, the disclosed composition is administered in an initial dose then at every 4 weeks thereafter. In some embodiments, the disclosed composition may be administered repetitively to 1, 2, 3, 4, 5, 6, or more times after the initial administration of the composition. In a typical embodiment, the disclosed composition is administered repetitively up to about 10 more times after the initial administration of the disclosed composition. In an alternative embodiment, the disclosed composition is administered more than about 10 times after the initial administration of the disclosed composition.

The disclosure relates to pharmaceutical compositions comprising therapeutically effective amounts of disclosed T-cells and a pharmaceutically acceptable carrier. In some embodiments, the disclosed compositions are administered to a subject in the form of a pharmaceutical composition, such as a composition comprising the cells or cell populations and a pharmaceutically acceptable carrier or excipient. The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc., or immunosuppressive agents, e.g., cyclosporin A, tacrolimus, mycophenolate, rapamycin, corticosteroids, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The choice of carrier in the pharmaceutical composition may be determined in part by the particular method used to administer the cell composition. Accordingly, there is a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21st ed. (May 1, 2005, incorporated by reference).

In some embodiments, the pharmaceutical composition comprises the disclosed composition in an amount that is effective to treat or prevent the disease or condition, such as a therapeutically effective or prophylactically effective amount. Thus, in some embodiments, the methods of administration include administration of the disclosed composition at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the disclosed composition is administered at a desired dosage, which in some aspects includes a desired dose or number of cells and/or a desired ratio of T-cell subpopulations. Thus, the dosage of cells in some embodiments is based on a total number of cells (or number per m² body surface area or per kg body weight) and a desired ratio of the individual populations or sub-types. In some embodiments, the dosage of cells is based on a desired total number (or number per m² body surface area or per kg of body weight) of cells in the individual populations or of individual cell types. In some embodiments, the dosage is based on a combination of such features, such as a desired number of total cells, desired ratio, and desired total number of cells in the individual populations.

In some embodiments, the disclosed composition is administered at or within a tolerated difference of a desired dose of total cells, such as a desired dose of T-cells. In some aspects, the desired dose is a desired number of cells, a desired number of cells per unit of body surface area or a desired number of cells per unit of body weight of the subject to whom the cells are administered, e.g., cells/m² or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m² or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and a desired ratio, and/or based on a desired fixed dose of two or more, e.g., each, of the individual T-cell subpopulations. Thus, in some embodiments, the dosage is based on a desired fixed or minimum dose of T-cell subpopulations and a desired ratio thereof.

In certain embodiments, the disclosed composition is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual T-cell subpopulations of cells is within a range of between at or about 104 and at or about 10⁹ cells/meter² (m²) body surface area, such as between 10⁵ and 10⁶ cells/m² body surface area, for example, at or about 1×10⁵ cells/m², 1.5×10⁵ cells/m², 2×10⁵ cells/m², or 1×10⁶ cells/m² body surface area. For example, in some embodiments, the cells are administered at, or within a certain range of error of, between at or about 104 and at or about 10⁹ T-cells/meter² (m²) body surface area, such as between 10⁵ and 10⁶ T-cells/m² body surface area, for example, at or about 1×10⁵ T-cells/m², 1.5×10⁵ T-cells/m², 2×10⁵ T-cells/m², or 1×10⁶ T-cells/m² body surface area.

In some embodiments, the cells are administered at or within a certain range of error of between at or about 104 and at or about 10⁹ cells/meter² (m²) body weight, such as between 10⁵ and 10⁶ cells/m² body weight, for example, at or about 1×10⁵ cells/m², 1.5×10⁵ cells/m², 2×10⁵ cells/kg, or 1×10⁶ cells/m² body surface area. In some embodiments, the cells are administered at or within a certain range of error of between at or about 10⁷ and at or about 5×10⁷ cells/m² body weight.

Product Release Testing and Characterization of T-cell subpopulations. Prior to infusion, the disclosed composition may be characterized for safety and release testing. Product release testing, also known as lot or batch release testing, is an important step in the quality control process of drug substances and drug products. This testing verifies that a T-cell subpopulation and/or disclosed composition meets a pre-determined set of specifications. Pre-determined release specifications for T-cell subpopulations and disclosed compositions include confirmation that the cell product is >70% viable, has <5.0 EU/ml of endotoxin, is negative for aerobic, anaerobic, fungal pathogens and mycoplasma, and lacks reactivity to allogeneic PHA blasts, for example, with less than about 10% lysis to PHA blasts. The phenotype of the disclosed composition may be determined with requirements for clearance to contain, in one non-limiting embodiment, <2% dendritic cells and <2% B cells. The HLA identity between the disclosed composition and the donor is also confirmed.

Antigen specificity of the T-cell subpopulations can be tested via an Interferon-γ Enzyme-Linked Immunospot (IFN-γ ELISpot) assay. Other cytokines can also be utilized to measure antigen specificity, including but not limited to, TNFα and IL-4. Pre-stimulated effector cells and target cells pulsed with the HPIV3 antigen of interest are incubated in a 96-well plate (pre-incubated with anti-INFγ antibody) at an E/T ratio of about 1:2. They are compared with non-HPIV3 antigen control, an irrelevant peptide not used for T-cell generation, and SEB as a positive control. After washing, the plates are incubated with a biotinylated anti-IFNγ antibody. Spots are detected by incubating with streptavidin-coupled alkaline phosphatase and substrate. Spot forming cells (SFCs) are counted and evaluated using an automated plate reader.

The phenotype of the disclosed composition can be determined by extracellular antibody staining with anti-CD3, CD4, CD8, CD45, CD19, CD16, CD56, CD14, CD45, CD83, HLA-DR, TCRαβ, TCRγδ and analyzed on a flow cytometer. Annexin-V and PI antibodies can be used as viability controls, and data analyzed with FlowJo Flow Cytometry software (Treestar, Ashland, Oreg., USA).

The lytic capacity of T-cell subpopulations may be evaluated via ⁵¹Chromium (⁵¹Cr) and Europium (Eu)-release cytotoxicity assays to test recognition and lysis of target cells pulsed with viral peptides by the T-cell subpopulations and disclosed compositions.

Typically, activated primed T-cells (effector cells) may be tested against ⁵¹Cr-labeled target cells at effector-to-target ratios of, for example, about 40:1, about 20:1, about 10:1, and about 5:1. Cytolytic activity can be determined by measuring ⁵¹Cr release into the supernatant on a gamma-counter. Spontaneous release is assessed by incubating target cells alone, and maximum lysis by adding 1% Triton X-100. Specific lysis was calculated as: specific lysis (%)=(experimental release−spontaneous release)/(maximum release−spontaneous release)×100.

Europium-release assays may also be utilized to measure the lytic capacity of T-cell subpopulations and disclosed compositions. This is a non-radioactive alternative to the conventional Chromium-51 (⁵¹Cr) release assay and works on the same principle as the radioactive assay Target cells are first loaded with an acetoxymethyl ester of BATDA. The ligand penetrates the cell membrane quickly. Within the cell, the ester bonds are hydrolyzed to form a hydrophilic ligand (TDA), which no longer passes through the cell membrane. If cells are lysed by an effector cell, TDA is released outside the cell into the supernatant. Upon addition of Europium solution to the supernatant, Europium can form a highly fluorescent and stable chelate with the released TDA (EuTDA). The measured fluorescence signal correlates directly with the number of lysed cells in the cytotoxicity assay. Specific lysis was calculated as: specific lysis (%)=(experimental release−spontaneous release)/(maximum release−spontaneous release)×100.

Monitoring. Following administration of the cells, the biological activity of the administered cell populations in some embodiments is measured, e.g., by any of a number of known methods. Parameters to assess include specific binding of a T-cell or other immune cell to antigen, in vivo, e.g., by imaging, or ex vivo, e.g., by ELISA or flow cytometry. In certain embodiments, the ability of the administered cells to destroy target cells may be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004), all incorporated herein by reference. In certain embodiments, the biological activity of the cells is measured by assaying expression and/or secretion of one or more cytokines, such as IFNγ, IL-2, and TNF. In some aspects, the biological activity is measured by assessing clinical outcome, such as reduction in disease symptoms or viral load.

Combination Therapies. In one aspect of the disclosure, the T-cell compositions disclosed herein can be beneficially administered in combination with another therapeutic regimen for beneficial, additive, or synergistic effects. In such combination therapies, the disclosed composition provides a HPIV3 or HPIV1 therapy to clear the HPIV3 or HPIV1 infecting the patient. In some embodiments, therefore, the disclosed composition is administered in combination with another therapy to treat an underlying disease or disorder. The second therapy can be a pharmaceutical or a biologic agent (for example an antibody) to increase the efficacy of treatment with a combined or synergistic approach.

In some embodiments, the disclosed composition described herein can be administered in combination with at least one agent or drug that is currently used for treating the same viral infection. For example, the disclosed composition described herein can be administered in combination with at least one agent or drug that is currently used for treating HPIV3 infection, e.g., Ribavirin or other antiviral agents. Likewise, when used to treat HPIV3, the disclosed composition described herein can be administered in combination with at least one agent or drug that is currently used for treating respiratory infections, diseases or disorders, such as croup, bronchiolitis and pneumonia, or airway inflammation.

In some embodiments, the disclosed composition described herein can be administered in combination with at least one anti-inflammatory agent. The anti-inflammatory agent can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cinhazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, toimetin, toimetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

In some embodiments, the disclosed composition described herein can be administered in combination with at least one immunomodulatory agent.

In some embodiments, the disclosed composition described herein can be administered in combination with at least one immunosuppressive agent. The immunosuppressive agent may be selected from the group consisting of a calcineurin inhibitor, e.g. a cyclosporin or an ascomycin, e.g. Cyclosporin A (NEORAL®), tacrolimus, a mTOR inhibitor, e.g. rapamycin or a derivative thereof, e.g. Sirolimus (RAPAMUNE®), Everolimus (Certican®), temsirolimus, biolimus-7, biolimus-9, a rapalog, e.g. azathioprine, campath 1H, a SIP receptor modulator, e.g. fingolimod or an analogue thereof, an anti-IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, 15-deoxyspergualin, tresperimus, Leflunomide ARAVA®, anti-CD25, anti-IL2R, Basiliximab (SIMULECT®), Daclizumab (ZENAPAX®), mizorbine, methotrexate, dexamethasone, pimecrolimus (Elidel®), abatacept, belatacept, etanercept (Enbrel®), adalimumab (Humira®), infliximab (Remicade®), an anti-LFA-1 antibody, natalizumab (Antegren®), Enlimomab, ABX-CBL, antithymocyte immunoglobulin, siplizumab, and efalizumab. Non-limiting examples of immunosuppressive agents that may be used in a combination therapy with the T-cell composition disclosed herein are provided in the table below.

| Medication | Typical dosage | | Notes |
|---|---|---|---|
| Cyclosporin A | 3-5 | mg/kg/day | |
| Tacrolimus | 0.15-0.4 | mg/kg/day | |
| Mycophenolate | 500-1000 | mg twice daily | |
| Rapamycin | 1-5 | mg/day | |
| Corticosteroids | 0.25-2 | mg/kg/day | Doses > 0.5 mg/kg/ |

| | |
|---|---|
| (prednisone or | day are likely to |
| equivalents) | inactivate VSTs |

Methods of Manufacturing Disclosed Compositions. T-cell subpopulations specific for a single HPIV3 antigen to be combined into the disclosed compositions for therapeutic administration described herein can be generated using any known method in the art or as described herein. Activated T-cell subpopulations that recognize at least one epitope of a HPIV3 antigen can be generated by any method known in the art or as described herein. Non-limiting exemplary methods of generating activated T-cell subpopulations that recognize at least one epitope of a HPIV3 antigen can be found in, for example Shafer et al., Leuk Lymphoma (2010) 51(5):870-880; Cruz et al., Clin Cancer Res., (2011) 17(22): 7058-7066; Quintarelli et al., Blood (2011) 117(12): 3353-3362; and Chapuis et al., Sci Transl Med (2013) 5(174):174ra27, all incorporated herein by reference.

Generally, generating the T-cell subpopulations of the disclosed compositions of the present disclosure may involve (i) collecting a peripheral blood mononuclear cell product from a donor; (ii) determining the HLA subtype of the mononuclear cell product; (iii) separating the monocytes and the lymphocytes of the mononuclear cell product; (iv) generating and maturing dendritic cells (DCs) from the monocytes; (v) pulsing the DCs with a HPIV3 antigen; (vi) optionally carrying out a CD45RA+ selection to isolate naïve lymphocytes; (vii) stimulating the naïve lymphocytes with the peptide-pulsed DCs in the presence of a cytokine cocktail; (viii) repeating the T-cell stimulation with fresh peptide-pulsed DCs or other peptide-pulsed antigen presenting cells in the presence of a cytokine cocktail; (ix) harvesting the T-cells and cryopreserving for future use.

In some aspects, generating the T-cell subpopulations of the disclosed compositions of the present disclosure may involve (i) collecting a peripheral blood mononuclear cell product from a donor; (ii) determining the HLA subtype of the mononuclear cell product; (iii) separating the monocytes and the lymphocytes of the mononuclear cell product; (iv) generating and maturing dendritic cells (DCs) from the monocytes, (v) pulsing the DCs with a HPIV3 antigen; (vi) optionally carrying out a CD45RA+ selection to isolate naïve T-cells; (vii) stimulating the naïve T-cells with the peptide-pulsed DCs in the presence of a cytokine cocktail; (viii) repeating the T-cell stimulation with fresh peptide-pulsed DCs or other peptide-pulsed antigen presenting cells in the presence of a cytokine cocktail; (ix) harvesting the T-cells and cryopreserving for future use.

In some embodiments, the T-cell subpopulation of the disclosed composition according to the present disclosure is primed and expanded for at least about 3, 5, 6, 7, 8, 9, 10, 11, 12, 13, 14, 15, 16, 17, 18, 19, or 20 days. In some embodiments, the T-cell subpopulation of the disclosed composition according to the present disclosure is primed and expanded for at least about 5-15 days. In some embodiments, the T-cell subpopulation of the disclosed composition according to the present disclosure is primed and expanded for at least about 10-12 days.

Collecting a Peripheral Blood Mononuclear Cell Product from a Donor. The generation of T-cell subpopulations to be specific to one or more HPIV3 antigens generally requires a peripheral blood mononuclear cell (PBMC) product from a donor, either an allogeneic or autologous donor, as a starting material. Isolation of PBMCs is well known in the art. Non-limiting exemplary methods of isolating PBMCs are provided in Grievink, H. W., et al. (2016) “Comparison of three isolation techniques for human peripheral blood mononuclear cells: Cell recovery and viability, population composition, and cell functionality,” Biopreservation and BioBanking, which is incorporated herein by reference. The PBMC product can be isolated from whole blood, an apheresis sample, a leukapheresis sample, or a bone marrow sample provided by a donor. In some embodiments, the starting material is an apheresis sample, which provides a large number of initially starting mononuclear cells, potentially allowing a large number of different T-cell subpopulations to be generated. In some embodiments, the PBMC product is isolated from a sample containing peripheral blood mononuclear cells (PBMCs) provided by a donor. In some embodiments, the donor is a healthy donor. In some embodiments, the PBMC product is originated from cord blood. In some embodiments, the donor is the same donor providing stem cells for a hematopoietic stem cell transplant (HSCT).

Determining HLA Subtype. When the T-cell subpopulations are generated from an allogeneic, healthy donor, the HLA subtype profile of the donor source is determined and characterized. Determining HLA subtype (i.e., typing the HLA loci) can be performed by any method known in the art. Non-limiting exemplary methods for determining HLA subtype can be found in Lange, V., et al., BMC Genomics (2014)15: 63; Erlich, H., Tissue Antigens (2012) 80:1-11; Bontadini, A., Methods (2012) 56:471476; Dunn, P. P., Int J Immunogenet (2011) 38:463-473; and Hurley, C. K., “DNA-based typing of HLA for transplantation.” in Leffell, M. S., et al., eds., Handbook of Human Immunology, 1997. Boca Raton: CRC Press, each independently incorporated herein by reference. Preferably, the HLA-subtyping of each donor source is as complete as possible.

In some embodiments, the determined HLA subtypes include at least 4 HLA loci, preferably HLA-A, HLA-B, HLA-C, and HLA-DRB1. In some embodiments, the determined HLA subtypes include at least 6 HLA loci. In some embodiments, the determined HLA subtypes include at least 6 HLA loci. In some embodiments, the determined HLA subtypes include all of the known HLA loci. In general, typing more HLA loci is preferable for practicing the disclosure, since the more HLA loci that are typed, the more likely the allogeneic T-cell subpopulations selected will have highest activity relative to other allogeneic T-cell subpopulations that have HLA alleles or HLA allele combinations in common with the patient or the diseased cells in the patient.

Separating the Monocytes and the Lymphocytes of the Peripheral Blood Mononuclear Cell Product. In general, the PBMC product may be separated into various cell-types, for example, into platelets, red blood cells, lymphocytes, and monocytes, and the lymphocytes and monocytes retained for initial generation of the T-cell subpopulations. The separation of PBMCs is known in the art. Non-limiting exemplary methods of separating monocytes and lymphocytes include Vissers et al., J Immunol Methods. 1988 Jun. 13; 110(2):203-7 and Wahl et al., Current Protocols in Immunology (2005) 7.6A.1-7.6A.10, which are incorporated herein by reference. For example, the separation of the monocytes can occur by plate adherence, by CD14+ selection, or other known methods. The monocyte fraction is generally retained in order to generate dendritic cells used as an antigen presenting cell in the T-cell subpopulation manufacture. The lymphocyte fraction of the PBMC product can be cryopreserved until needed, for example, aliquots of the lymphocyte fraction (˜5×10⁷ cells) can be cryopreserved separately for both Phytohemagglutinin (PHA) Blast expansion and T-cell subpopulation generation.

Generating Dendritic Cells. The generation of mature dendritic cells used for antigen presentation to prime T-cells is well known in the art. Non-limiting exemplary methods are included in Nair et al., “Isolation and generation of human dendritic cells.” Current protocols in immunology (2012) 0 7: Unit 7.32. doi:10.1002/0471142735.im0732s99 and Castiello et al., Cancer Immunol Immunother, 2011 April; 60(4):457-66, which are incorporated herein by reference. For example, the monocyte fraction can be plated into a closed system bioreactor such as the Quantum Cell Expansion System, and the cells allowed to adhere for about 2-4 hours at which point 1,000 U/mL of IL-4 and 800 U/mL GM-CSF can be added. The concentration of GM-CSF and IL-4 can be maintained. The dendritic cells can be matured using a cytokine cocktail. In some embodiments the cytokine cocktail consists of LPS (30 ng/mL), IL-4 (1,000 U/mL), GM-CSF (800 U/mL), TNF-Alpha (10 ng/mL), IL-6 (100 ng/mL), and IL-1beta (10 ng/mL). The dendritic cell maturation generally occurs in 2 to 5 days. In some embodiments, the adherent DCs are harvested and counted using a hemocytometer. In some embodiments, a portion of the DCs are cryopreserved for additional further stimulations.

Pulsing the Dendritic Cells. The non-mature and mature dendritic cells are pulsed with one or more peptides of one or more HPIV3 antigens. In some embodiments, the HPIV3 antigenic peptides used to pulse the non-mature and mature dendritic cells are from a pool or library of overlapping peptide fragments of the HPIV3 antigen, as provided for example, in commercially available overlapping peptide libraries. In some embodiments, the pool or library of overlapping peptide fragments of the HPIV3 antigen comprises peptide fragments of from about 10 to about 20 amino acids in length, for example, 15-mers peptide fragments containing 9, 10 or 11 amino acids of overlap between each sequence formed. In some embodiments, the HPIV3 peptides used to pulse the non-mature and mature dendritic cells are from specifically selected HLA-restricted peptides generated by determining the HLA profile of the donor source, and including peptide epitopes derived from the targeted HPIV3 peptide that are active through the donor's HLA type. Methods of pulsing a dendritic cell with a HPIV3 peptides are known. For example, about 100 ng of one or more HPIV3 peptides, for example a peptide library, can be added per 10 million dendritic cells and incubated for about 30 to 120 minutes. Naïve T-cell Selection of Lymphocytes. In order to increase the potential number of specific HPIV3 antigen activated T-cells and reduce T-cells that target other antigens, it may be preferable to utilize naïve T-cells as a starting material. To isolate naïve T-cells, the lymphocytes can undergo a selection, for example, CD45RA+ cells selection. CD45RA+ cell selection methods are generally known in the art. Non-limiting exemplary methods are found in Richards et al., Immune memory in CD4+CD45RA+ T cells. Immunology. 1997; 91(3):331-339 and McBreen et al., J Virol. 2001 May; 75(9): 4091-4102, which are incorporated herein by reference. For example, to select for CD45RA⁺ cells, the cells can be labeled using 1 vial of CD45RA microbeads from Miltenyi Biotec per 1×10¹¹ cells after 5-30 minutes of incubation with 100 mL of CliniMACS buffer and approximately 3 mL of 10% human IVIG, 10 ug/mL DNAase I, and 200 mg/mL of magnesium chloride. After 30 minutes, cells will be washed sufficiently and resuspended in 20 mL of CliniMACS buffer. The bag will then be set up on the CLINIMACS Plus device and the selection program can be run according to manufacturer's recommendations. After the program is completed, cells can be counted, washed and resuspended in “CTL Media” consisting of 44.5% EHAA Click's, 44.5% Advanced RPMI, 10% Human Serum, and 1% GlutaMAX.

Stimulating Naïve T cells with Peptide-Pulsed Dendritic Cells. Prior to stimulating naïve T-cells with the dendritic cells, it may be preferable to irradiate the DCs, for example, at 25 Gy. The DCs and naïve T-cells are then co-cultured. The naïve T-cells can be co-cultured in a ratio range of DCs to T-cells of about 1:5-1:50, for example, about 1:5; about 1:10, about 1:15, about 1:20, about 1:25, about 1:30, about 1:35, about 1:40, about 1:45, or about 1:50. The DCs and T-cells are generally co-cultured with cytokines. In some embodiments, the cytokines are selected from a group consisting of IL-6 (100 ng/mL), IL-7 (10 ng/mL), IL-15 (5 ng/mL), IL-12 (10 ng/mL), and IL-21 (10 ng/mL).

Second T-Cell Stimulation. In general, it is preferable to further stimulate the T-cell subpopulations with one or additional stimulation procedures. The additional stimulation can be performed with, for example, fresh DCs pulsed with the same peptides as used in the first stimulation, similarly to as described above. In some embodiments, the cytokines used during the second stimulation are selected from a group consisting of IL-7 (10 ng/mL) and IL-2 (100 U/mL).

Alternatively, peptide-pulsed PHA blasts can be used as the antigen presenting cell. The use of peptide-pulsed PHA blasts to stimulate and expand T-cells are well known in the art. Non-limiting exemplary methods can be found in Weber et al., Clin Cancer Res. 2013 Sep. 15; 19(18): 5079-5091 and Ngo et al., J. Immunother. 2014 May; 37(4): 193-203, which are incorporated herein by reference. The peptide-pulsed PHA blasts can be used to expand the T-cell subpopulation in a ratio range of PHA blasts to expanded T cells of about 10:1-1:10. For example, the ratio of PHA blasts to T-cells can be about 10:1, between about 10:1 and about 9:1, between about 9:1 and about 8:1, between about 8:1 and about 7:1, between about 7:1 and about 6:1, between about 6:1 and about 5:1, between about 5:1 and about 4:1, between about 4:1 and about 3:1, between about 3:1 and about 2:1, between about 2:1 and about 1:1, between about 1:1 and about 1:2, between about 1:2 and about 1:3, between about 1:3 and about 1:4, between about 1:4 and about 1:5, between about 1:5 and about 1:6, between about 1:6 and about 1:7, between about 1:7 and about 1:8, between about 1:8 and about 1:9, between about 1:9 and about 1:10. In general, cytokines are included in the co-culture, and are selected from the group consisting of IL-7 (10 ng/mL) and IL-2 (100 U/mL).

Additional T-Cell Expansion and T-Cell Subpopulation Harvest Additional T-cell stimulations may be necessary to generate the necessary number of T-cell subpopulations for use in the disclosed composition. Following any stimulation and expansion, the T-cell subpopulations are harvested, washed, and concentrated. In some embodiments, a solution containing a final concentration of 10% dimethyl sulfoxide (DMSO), 50% human serum albumin (HSA), and 40% Hank's Balanced Salt Solution (HBSS) will then be added to the cryopreservation bag. In some embodiments, the T-cell subpopulation will be cryopreserved in liquid nitrogen.

Further Characterization of the T-cell Subpopulation. The T-cell subpopulations for use in the disclosed composition of the present disclosure are HLA-typed and can be further characterized prior to use or inclusion in the disclosed composition. For example, each of the T-cell subpopulations may be further characterized by, for example, one or more of i) determining the HPIV3 specificity of the T-cell subpopulation; ii) identifying the HPIV3 antigen epitope(s) the T-cell subpopulation is specific to; iii) determining whether the T-cell subpopulation includes MHC Class I or Class II restricted subsets or a combination of both; iv) correlating antigenic activity through the T-cell's corresponding HLA-allele; and v) characterizing the T-cell subpopulation's immune effector subtype concentration, for example, the population of effector memory cells, central memory cells, γδ T-cells, CD8+, CD4+, NKT-cell.

Determining the HPIV3 Antigen Specificity of the T-Cell Subpopulation. The T-cell subpopulations of the disclosed composition can be further characterized by determining each T-cell subpopulation's specificity for its targeted HPIV3 antigen. Specificity can be determined using any known procedure, for example, an ELISA based immunospot assay (ELISpot). In some embodiments, HPIV3 antigen specificity of the T-cell subpopulation is determined by ELISpot assay. ELISpot assays are widely used to monitor adaptive immune responses in both humans and animals. The method was originally developed from the standard ELISA assay to measure antibody secretion from B cells (Czerkinsky C. et al. (1983) A solid-phase enzyme-linked immunospot (ELISPOT) assay for enumeration of specific antibody-secreting cells. J. Immunol Methods 65: 109-21), which is incorporated herein by reference. The assay has since been adapted to detect secreted cytokines from T cells, for example IFN-γ, and is an essential tool for understanding the helper T cell response.

A T-cell ELISpot assay generally comprises the following steps:

i) a capture antibody specific for the chosen analyte, for example IFN-γ, is coated onto a PVDF plate;

ii) the plate is blocked, usually with a serum;

iii) the T-cell subpopulation is added along with the specific, targeted HPIV3 antigen;

iv) plates are incubated and secreted cytokines, for example IFN-γ, are captured by the immobilized antibody on the PVDF surface;

v) after washing, a biotinylated detection antibody is added to allow detection of the captured cytokine, and

vi) the secreted cytokine is visualized using an avidin-HRP or avidin-ALP conjugate and a colored precipitating substrate.

Each colored spot represents a cytokine secreting cell. The spots can be counted by eye or by using an automated plate-reader. Many different cytokines can be detected using this method including IL-2, IL-4, IL-17, IFN γ, TNFα, and granzyme B. The size of the spot is an indication of the per cell productivity and the avidity of the binding. The higher the avidity of the T cell recognition the higher the productivity resulting in large, well-defined spots.

Identifying the HPIV3 Epitope(s) the T-Cell Subpopulation is Specific to a Subject. The T-cell subpopulations of the disclosed composition can be further characterized by identifying the HPIV3 epitope(s) the T-cell subpopulation that is specific to a subject. General methods for identifying epitope(s) to which a T-cell population recognizes may include testing of overlapping peptide libraries encompassing HPIV3 antigens, in which the test utilized could include IFN-γ ELISpot, intracellular flow cytometry, or cytotoxicity assays via CD107a expression or 51-chromium release assay.

Determining the T-cell Subpopulation's MHC-Class I or Class H Restricted Subsets. The T-cell subpopulations of the disclosed composition can be further characterized by determining the subpopulation's MHC Class I or Class II subset restriction response. This is done to determine whether epitope recognition is mediated by CD8+(class I) or CD4+(class II) T-cells. General methods for determining the MHC Class I or Class II response are generally known in the art. A non-limiting exemplary method is found in Weber et al., Clin Cancer Res. 2013 Sep. 15; 19(18): 5079-5091, which is incorporated herein by reference. For example, to determine HLA restriction response, T-cells can be pre-incubated with class I or II blocking antibodies for 1 hour before the addition of antigen peptides in an ELISPOT assay using autologous peptide-pulsed PHA blasts as targets with unpulsed PHA blasts as a control. IFNγ-secretion is measured in the presence of each blocking antibody. If, when pre-incubated with a class I blocking antibody, IFNγ-secretion is reduced to background levels then this is indicative of a class I restriction and the epitope recognition is mediated by CD8+ T cells. If, when pre-incubated with a class I blocking antibody, IFNγ-secretion is reduced to background levels then this is indicative of a class H restriction and the epitope recognition is mediated by CD4+ T cells.

The direct detection of antigen-specific T cells using tetramers of soluble peptide-major histocompatibilty complex (pMHC) molecules is widely used in both basic and clinical immunology. Tetrameric complexes of HLA molecules can be used to stain antigen-specific T cells in FACS analysis. In vitro synthesized soluble HLA-peptide complexes are used as tetrameric complexes to stain antigen specific T-cells in FACS analysis (Altman et al., Science 274: 94-96, 1996). T-cell subpopulations specific for HPIV3 antigens are stained with CD8 fluorescein isothiocyanate (FITC) and with phycoerythrin (PE)-labeled MHC pentamers at various timepoints during in vitro stimulation. Antigen specificity is measured by flow cytometry.

Correlating Antigenic Activity through the T-Cell's Corresponding HLA-Allele. The T-cell subpopulation can be further characterized by correlating antigenic activity through the T-cell subpopulation's corresponding HLA-allele. Correlating antigenic activity through the corresponding HLA-allele can be done using any known method. For example, in some embodiments, a HLA restriction assay is used to determine antigen activity through a corresponding allele. Methods to determine T-cell restriction are known in the art and involve inhibition with locus specific antibodies, followed by antigen presentation assays (ELISPOT) with panels of cell lines matched or mismatched at the various loci of interest (see, e.g., (Oseroff et al., J Immunol (2010) 185(2): 943-955; Oseroff et al., J. Immunol (2012) 189(2): 679-688; Wang, Curr Protocols in Immunol (2009) Chap. 20, page 10; Wilson et al., J. Virol. (2001) 75(9): 4195-4207), each independently incorporated herein by reference. Because epitope binding to HLA class II molecules is absolutely necessary (but not sufficient) for T cell activation, data from in vitro HLA binding assays has also been useful to narrow down the possible restrictions (Arlehamn et al., J Immunol (2012b) 188(10):5020-5031). This is usually accomplished by testing a given epitope for binding to the specific HLA molecules expressed in a specific donor and eliminating from further consideration HLA molecules to which the epitope does not bind. To determine the HLA restriction of the identified epitope, T cells can be plated in an IFN-γ ELISPOT assay with HPIV3 antigen peptide pulsed PHA blasts that match at a single allele, measuring the strongest antigen activity, and identifying the corresponding allele.

Characterizing the T-cell Subpopulation's Immune Effector Subtype Concentration. The T-cell subpopulation is likely to be made up of different lymphocytic cell subsets, for example, a combination of CD4- T-cells, CD8+ T-cells, CD3+/CD56+ Natural Killer T-cells (CD3+ NKT), and TCR γδ T-cells (γδ T-cells). In particular, the T-cell subpopulation likely include at least CD4+ T-cells and CD8+ T-cells that have been primed and are capable of targeting a single specific HPIV3 antigen for infected cell killing and/or cross presentation. The T-cell subpopulation may further comprise activated γδ T-cells and/or activated CD3+/CD56+ NKT cells capable of mediating anti-HPIV3 responses. Accordingly, the T-cell subpopulation may be further characterized by determining the population of various lymphocytic subtypes, and the further classification of such subtypes, for example, by determining the presence or absence of certain clusters of differentiation (CD) markers, or other cell surface markers, expressed by the cells and determinative of cell subtype.

In some embodiments, the T-cell subpopulation may be analyzed to determine CD8+ T-cell population, CD4+, T-cell population, γδ T-cell population, NKT-cell population, and other populations of lymphocytic subtypes. For example, the population of CD4+ T-cells within the T-cell subpopulation may be determined, and the CD4+ T-cell subtypes further determined. For example, the CD4+ T-cell population may be determined, and then further defined, for example, by identifying the population of T-helper 1 (Th₁), T-helper 2 (Th₂), T-helper 17 (Th₁₇), regulatory T cell (T_{reg}), follicular helper T-cell (T_{fh}), and T-helper 9 (Th₉). Likewise, the other lymphocytic subtypes comprising the T-cell subpopulation can be determined and further characterized.

In addition, the T-cell subpopulation can be further characterized, for example, for the presence, or lack thereof, of one or more markers associated with, for example, maturation or exhaustion. T cell exhaustion (Tₑₓ) is a state of dysfunction that results from persistent antigen and inflammation. Tₑₓ cell populations can be analyzed using multiple phenotypic parameters, either alone or in combination. Hallmarks commonly used to monitor T cell exhaustion are known in the art and include, but are not limited to, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains).

The T-cell subpopulations of the described compositions described herein can be subjected to further selection, if desired. For example, a particular T-cell subpopulation for inclusion in a disclosed composition described herein can undergo further selection through depletion or enriching for a sub-population. For example, following priming, expansion, and selection, the cells can be further selected for other cluster of differentiation (CD) markers, either positively or negatively. For example, following selection of for example CD4+ T-cells, the CD4+ T-cells can be further subjected to selection for, for example, a central memory T-cells (T_{cm}). For example, the enrichment for CD4+T_{cm} cells comprises negative selection for cells expression a surface marker present on naïve T cells, such as CD45RA, or positive selection for cells expressing a surface marker present on T_{cm} cells and not present on naïve T-cells, for example CD45RO, CD62L, CCR7, CD27, CD127, and/or CD44. In addition, the T-cell subpopulations described herein can be further selected to eliminate cells expressing certain exhaustion markers, for example, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains).

Methods for characterizing lymphocytic cell subtypes are well known in the art, for example flow cytometry, which is described in Pockley et al., Curr Protoc Toxicol. 2015 Nov. 2; 66:18.8.1-34, which is incorporated herein by reference.

Identifying the disclosed composition Most Suitable for Administration Characterization of each T-cell subpopulation composition allows for the selection of the most appropriate T-cell subpopulations for inclusion in the disclosed composition for any given patient. The goal is to match the product with the patient that has the both the highest HLA match and greatest HPIV3 activity through the greatest number of shared alleles. In some embodiments, the T-cell subpopulation has at least one shared allele or allele combination with HPIV3 activity through that allele or allele combination. In some embodiments, the T-cell subpopulation has greater than 1 shared allele or allele combination with HPIV3 activity through that allele or allele combination. In some embodiments, the I-cell subpopulation with the most shared alleles or allele combinations and highest specificity through those shared alleles and allele combinations is provided to a human in need thereof. For example, if T-cell subpopulation 1 is about ⅝ HLA match with the patient with HPIV3 activity through 3 shared alleles or allele combinations while T-cell subpopulation 2 is about 6/8 HLA match with the patient with HPIV3 activity through 1 shared allele the skilled practitioner would select T-cell subpopulation 1 as it has HPIV3 activity through a greater number of shared alleles.

Banked T-Cell Subpopulations Directed to HPIV3 Antigens. The establishment of a T-cell subpopulation bank comprising discrete, characterized T-cell subpopulations for selection and inclusion in a disclosed composition bypasses the need for an immediately available donor and eliminates the wait required for autologous T cell production. Preparing T-cell subpopulations directed to specific HPIV3 antigens by using donors, for example healthy volunteers or umbilical cord blood, allows the production and banking of T-cell subpopulations readily available for administration. Because the T-cell subpopulations are characterized, the selection of suitable T-cell subpopulations can be quickly determined based on minimal information from the patient, for example HLA-subtype and, optionally HPIV3 expression profile.

From a single donor, a T cell composition can be generated for use in multiple patients who share HLA alleles that have activity towards a specific HPIV3 antigen. The T-cell subpopulation bank of the present disclosure includes a population of T-cell subpopulations which have been characterized as described herein. For example, the T-cell subpopulations of the bank are characterized as to HLA-subtype and one or more of i) HPIV3 specificity of the T-cell subpopulation; ii) HPIV3 epitope(s) the T-cell subpopulation is specific to; iii) T-cell subpopulation MHC Class I and Class II restricted subsets; iv) antigenic activity through the T-cell's corresponding HLA-allele; and v) immune effector subtype concentration, for example, the population of effector memory cells, central memory cells, γδ T-cells, CD8+, CD4+, NKT-cell.

In some embodiments, the present disclosure provides a method of generating a T-cell subpopulation bank comprising: (i) obtaining eligible donor samples; (ii) generating T-cell subpopulations specific to a single HPIV3 antigen; (iii) characterizing the T-cell subpopulation; (iv) cryopreserving the T-cell subpopulation; and (v) generating a database of T-cell subpopulation composition characterization data. In some embodiments, the T-cell subpopulations are stored according to their donor source. In some embodiments, the T-cell subpopulations are stored by HPIV3 antigen specificity. In some embodiments, the T-cell subpopulations are stored by human leukocyte antigen (HLA) subtype and restrictions.

The banked T-cell subpopulations described herein are used to comprise a disclosed composition for administration to a HPIV3-infected patient following the determination of the patient's HLA subtype and, optionally, HPIV3 antigen expression profile of the infected HPIV3 strain.

Aspects and embodiments of the present disclosure will now be illustrated, by way of example, with reference to the accompanying tables and figures. Further aspects and embodiments will be apparent to those skilled in the art. All documents mentioned in this text are incorporated herein by reference.

### EXAMPLES

### Example 1. Preparation of HPIV3-Specific T-Cells and Identification of Immunodominant HPIV3 Epitopes

Donors. Peripheral blood mononuclear cells (PBMCs) from healthy volunteers were obtained from Children's National Medical Center (Washington, D.C.) and the National Institutes of Health (Bethesda, Md.) under informed consent approved at each institution in accordance with the Declaration of Helsinki. PBMC were isolated by Ficoll/Hypaque centrifugation.

Peptides. For stimulation, custom-ordered pepmixes (peptide libraries of 15-mers overlapping by 10 amino acids) were used that encompassed one or more HPIV3 antigens selected from HN, Fus, mat, NP, P and L (JPT, Berlin, Del.). Protein sequences from the HPIV3 Wash/47885/57 strain were utilized for peptide production (Stokes et al., Virus Res. 1992; 25(1-2):91-103).

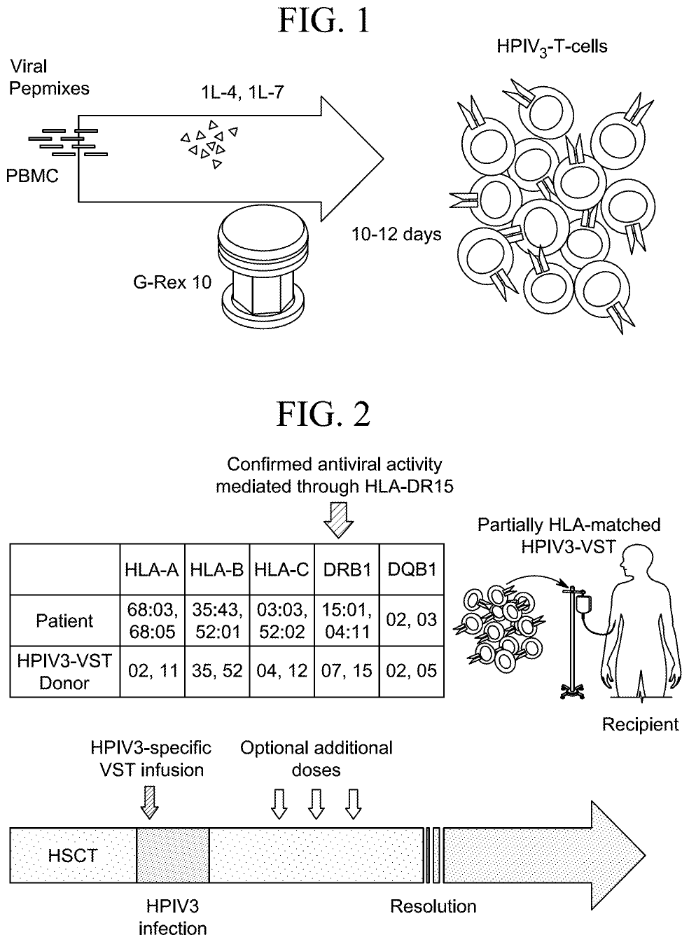

Generation of HPIV3-specific VST. PBMCs were pulsed with HPIV3 pepmixes pooled or individually (200 ng/peptide/15×10⁶ PBMCs) for 30 minutes at 37° C. Pulsed PBMC were cultured in Grex-10 bioreactors (Wilson Wolf New Brighton, Minn.) or 24-well plates, with medium containing 45% RPMI (GE Healthcare, Logan, Utah), 45% Click's medium (Irvine Scientific, Santa Ana, Calif.), 10% fetal bovine serum, and supplemented with 2 mM L-glutamine, IL-4 at 400 IU/ml (R&D Systems, Minneapolis, Minn.), and IL-7 at 10 ng/ml (R&D Systems, Minneapolis, Minn.). VSTs were supplemented with cytokines at day 4 and medium at day 6-7, and were harvested between days 10-12. FIG. 1 generally describes generation of HPIV3-specific T cells.

IFN-γ enzyme-linked immunospot (ELISpot) assay. Antigen specificity of T-cells was measured with IFN-γ ELISpot (Millipore, Burlington, Mass.). Ninety-six-well filtration plates (MultiScreen, MSIPS4W10, Millipore, Billerica, Mass.) were coated overnight with 10 μg/ml anti-hu-IFN-mAb (capture mAB (1-DIK purified); Mabtech). VST (1×10⁵ cells/well) or PBMC (2×10⁵ cells/well) were plated in ninety-six well plates and stimulated with overlapping 15-mer peptide pools encompassing HPIV3 antigen(s) at 0.5 nmol/peptide/well. Each condition was run in duplicate or triplicate. Staphylococcal enterotoxin-B (SEB) was used as a positive control (1 μg/ml). A 15-mer peptide pool encompassing the actin protein was used as a negative control. After 18-24 hours, the plates were washed and incubated with the secondary biotin-conjugated anti-human-IFNγ-mAb (detector mAb (7-B6-1 biotin); Mabtech, Cincinnati, Ohio) at 1 μg/ml. After incubation with avidin:biotinylated HRP complex (Vectastain Elite ABC Kit (standard), PK6100; Vector Laboratories, Burlingame, Calif.), plates were developed with AEC substrate (A6926, Sigma-Aldrich, St Louis, Mo.), dried overnight, and quantified (Zellnet Consulting, New York, N.Y.). The frequency of T-cells specific for each peptide was expressed as spot-forming cells (SFC) per 1×10⁵ cells (or 2×10⁵ cells for PBMC conditions). A correction for confluence was applied as follows: Well count+2×(Well count×(% confluence/[1−% confluence])). Blocking of MHC class I and class II was performed by incubation of T-cells with mouse polyclonal antibodies to human HLA-A/B/C or HLA-DR/DP/DQ (Dako, Carpinteria, Calif.) for 1 hour prior to plating for ELISpot assay as described above. Responses that were at least 20 SFC/1×10⁵ cells greater than background (unstimulated VSTs plated with media alone) were regarded as significant.

Flow cytometry. Staining of cell surface markers on PBMC and T-cells was performed with CD3-APC/Vio770, CD4-Vioblue, CD8-Viogreen, CD19-FITC, CD56-PE/Vio770, CD16-PE, CD62L-Vioblue, CD45RA-PE, CD45RO-APC, and CCR7-FITC (Miltenyi, San Diego, Calif.; BD, Franklin Lakes, N.J.). All samples were acquired on a MACSQuant Cytometry (Miltenyi) and the data analyzed with Flow Jo (Treestar, Ashland, Oreg.).

Intracellular cytokine staining. T-cells were rested with IL-2 (100 U/ml) overnight and then stimulated with pepmixes encompassing viral or control proteins (JPT) at 0.5 nmol/ul, along with anti-CD49d/CD28 antibodies (BD) at 1:1000 for co-stimulation and Brefeldin A at 1 μl/ml. SEB and Actin were utilized as controls. T-cells were stained with cell surface markers (CD3, CD4, CD8), followed by permeabilization with Cytofix/Cytoperm (BD FastImmune), washing, and staining with IFN-γ-APC (Biolegend, San Diego, Calif.), IL-2-FITC, and TNFα-PE (BD).

CD107A mobilization. For evaluation of CD107a mobilization, T-cells were stimulated with viral or control pepmixes (JPT) in the presence of Brefeldin A at 1 μl/ml for 4 hours, followed by surface marker staining utilizing the antibodies listed above. Samples were acquired on MACSQuant Analyzer 10 (Miltenyi Biotec) and analyzed with Flow Jo (Treestar, Ashland, Oreg.).

Interferon-γ secretion assay. Measurement of IFN-γ secretion by HPIV3-specific T-cells was performed in cells that were stimulated for 4 hours with or without 2.5 μl/ml HPIV3 pepmixes in presence of 1 μl/ml CD49d/CD28 antibodies. Cell surface detection of IFN-γ secreted molecules was performed with an IFN-γ specific high affinity capture matrix, subsequently stained with PE-anti IFN-γ, and magnetically labeled with anti-PE microbeads following manufacturer recommendations (Miltenyi Biotec). PE-IFN-γ+ cells were enriched and analyzed with a MACSQuant Analyzer 10 (Miltenyi Biotec). Cell surface phenotype analysis was performed via CD4 and CD8 VioBlue; CD45RA-APC VioGreen; CCR7 APC; and CD62L-FITC mAbs (Miltenyi Biotec).

Cytotoxicity assay. Cytotoxicity was measured by standard ⁵¹Cr release assay. Briefly, autologous or allogeneic phytohemagglutinin (PHA)-stimulated lymphoblasts were produced, pre-incubated with IFNγ (100 ng/ml) alone or with HPIV3 pepmixes (0.5 nmol/peptide/ml of each protein) for 12 hours, and then pulsed with ⁵¹Cr (Perkin Elmer, Waltham, Mass.) at 10 uCi per 1×10⁵ cells for 1 hour and washed. Effector T-cells were pre-incubated overnight with IL-2 (100 u/ml) and then plated with radiolabeled target cells at multiple effector to target ratios. Maximum release was determined by lysis of radiolabeled targets with 1% Triton-X100 detergent (Sigma-Aldrich). Targets and effectors were incubated at 37° C. for 4-6 hours, centrifuged, and supernatants transferred to a 96-well scintillation plate and allowed to dry. Plates were read on a MicroBeta2 LumiJet Scintillation counter (Perkin Elmer). Specific lysis was determined as follows: (Experimental counts per minute [CPM]−Background CPM)/(Maximum CPM−Background CPM). All conditions were performed in triplicate.

Multiplex cytokine assay. VST were plated on 96 well plates at 1×10⁵ cell per well and stimulated with HPIV3 pepmixes or controls (SEB, Actin) at the same conditions as described for IFNγ ELISpot, and incubated overnight at 37° C., 5% CO₂. Suspensions underwent centrifugation, and supernatants were removed for analysis. Cytokine analysis of the supernatants was performed using a custom Bio-Plex Pro kit (Bio-Rad, Hercules, Calif.) per manufacturer's protocol, and was analyzed on a Bio-Plex MAGPIX Multiplex Reader (Luminex, Madison, Wis.) using Bio-Plex Manager 6.1.

Data Analysis/Statistics. Results were evaluated using descriptive statistics (means, standard deviations, ranges). Comparative analysis between ELISpot and flow cytometry results for different epitopes was performed via one-way ANOVA. Comparisons between responses to viral peptides and irrelevant peptides were performed using a 2-sided unpaired T-Test. Analysis was performed in Graphpad Prism (GraphPad software, La Jolla. Calif.) and StatPlus (AnalystSoft, Walnut, Calif.).

Results. Several novel immunodominant epitopes within HPIV3 Matrix protein were identified within 10 different T-cell products from healthy donors. Products demonstrated antiviral activity by producing IFN-γ and TNF-α when stimulated with peptide. VSTs were polyfunctional and comprised of both CD4+ and CD8+ T-cells, though preliminary flow cytometry data shows that these epitopes are predominantly HLA Class II restricted. The epitopes identified are listed below in Table 1.

TABLE 1
T-cell epitopes with HPIV3 matrix protein

| HPIV3 Matrix | | |
|---|---|---|
| Epitope | Sequence | SEQ ID NO: |
| Peptide 38 | MLFDANKVALAPQCL | 7 |
| Peptide 39 | ANKVALAPQCLPLDR | 8 |
| Peptide 50 | SLPSTISINLQVHIK | 9 |
| Peptide 59 | LNFMVHLGLIKRKVG | 10 |
| Peptide 60 | VHLGLIKRKVGRMYS | 11 |
| Peptide 75 | EICYPLMDLNPHINL | 12 |
| Peptide 76 | PLMDLNPHLNLVIWA | 13 |
| Peptide 77 | LNPHLNLVIWASSVE | 14 |
| Peptide 78 | LNLVIWASSVEITRV | 15 |
| Peptide 82 | AIFQPSLPGEFRYYP | 16 |
| Peptide 83 | PSLPGEFRYYPNIIA | 17 |
| Peptide 84 | GEFRYYPNIIAKGVG | 18 |
| Peptide 85 | YYPNIIAKGVGKIKQ | 19 |

Usage of these epitopes could foreseeable allow third-party treatment of patients with HPIV3 infections with partially-HLA matched virus-specific T-cells targeting Matrix protein through one or more of these class II restricted epitopes. This would allow rapid treatment of patients with severe viral infection, thereby eliminating the need to produce customized T-cell products, and improving the feasibility of T-cell therapy against HPIV3. Accordingly, these epitopes and T cell responses to them were further characterized in the examples which follow.

### Example 2. HPIV3-Specific T-Cells Comprise Polyclonal Populations with a CD4 Effector T Cell Predominance

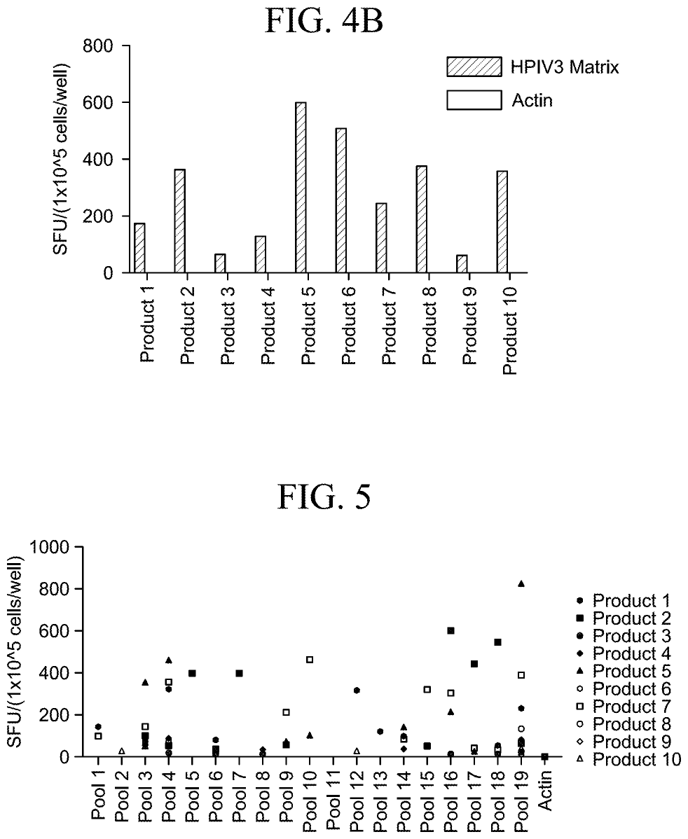

The inventors characterized the phenotypes of virus-specific T cells (VST). As shown by FIG. 4A, phenotyping data of the hexaviral-specific T cell products specific for cytomegalovirus (CMV), Epstein Barr virus (EBV), AdV, HHV6, BKV, and HPIV3 (T cell product numbers 1-4, 6, 8-10) and monoviral-specific T cell products specific only for HPIV3 (T cell product numbers 5, 7) was obtained using flow cytometry. Phenotypes, including memory differentiation status panel, are reported as % of total CD3+ cells. Product #5 was unavailable for memory/differentiation phenotyping. The definitions of the phenotypes are as follows: Naïve: CD45RA+CD45RO−CCR7+CD62L+CD95-Stem Cell Memory: CD45RA+CD45RO−CCR7+CD62L+CD95+ Central Memory: CD45RA-CD45RO+CCR7+CD62L+ Effector Memory: CD45RA−CD45RO+CCR7−CD62L− Terminal Effector Memory: CD45RA−CD45RO−CCR7−CD62L−.

FIG. 4B shows the overall HPIV3 Matrix specificity by product. All 10 viral-specific T cell products were stimulated with HPIV3 pepmix. Response was measured as spot forming units per well (SFU/1×10⁵ cells) by anti-IFN-g ELISpot assay. Unstimulated T-cells (CTL only) and stimulation with actin pepmix (irrelevant antigen) were used as negative controls.

The hexaviral and monoviral HPIV3-expanded populations primarily consisted of T-cells (CD3+ median 95.75%, range 76.3%-98.9%). CD4+ T-cells comprised the majority of the expanded T-cells (median 63.6%; range 45.3%-83.7%) while CD8+ T-cells made up lesser proportions (median 30%; range 11.4%-45.1%).

Differentiation status phenotyping of the CD3+ cells showed a predominance of effector memory cells (median 66%, range 29.8%-92.8%). All 10 T cell products showed specificity for HPIV3 Matrix protein (Average IFN-gamma spot forming units per well (SFU) 319.8/1×10⁵ cells) versus actin, an irrelevant antigen used as negative control. (SFU 0.5/1×10⁵ cells) (FIG. 4B). In silico testing (Immune Epitope Database, IEDB.org) predicted that all 10 products would have MHC class II restricted responses primarily mediated through HLA-DRB1 alleles (Table 3), and when stimulated with Matrix peptides, all products demonstrated a CD4+ response.

### Example 3. HPIV3-Specific CD4+ T-Cells Respond to Multiple Class 11-Restricted Epitopes within Matrix Protein

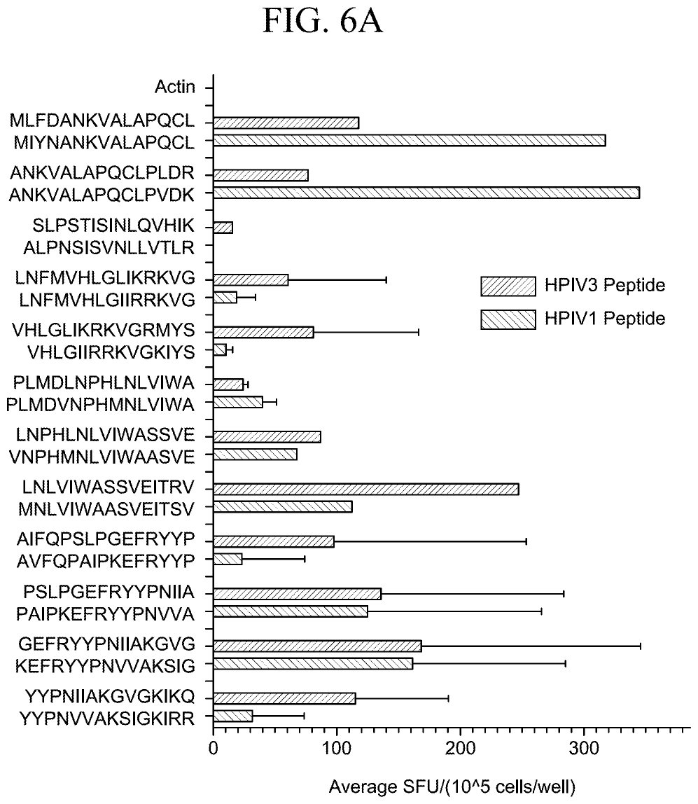

Eighteen of the 19 pools were recognized by at least one of the VST products (SFU>10/1×10⁵ cells), and three of the pools were recognized by five or more VST products (FIG. 5). Twelve novel epitopes were recognized among the 10 different T cell products 38, 39, 50, 59, 60, 76, 77, 78, 82, 83, 84, 85 (Table 3). All 10 T cell products recognized peptide 84 (GEFRYYPNIIAKGVG, SEQ ID NO: 18); median SFU 151.9/1×10⁵ cells (range 24-564); mean SFU 184.9/1×10⁵ cells); nine products recognized peptide 83 (PSLPGEFRYYPNIIA (SEQ ID NO: 17); median SFU 79.8/1×10⁵ cells (range 36-484); mean SFU 141.1/1×10⁵ cells); and seven products recognized peptide 82 (AIFQPSLPGEFRYYP, SEQ ID NO: 16); median SFU 43/1×10⁵ cells (range 22-443); mean SFU 107.1/1×10⁵ cells). The remaining nine epitopes were recognized by one to three products (Table 4). None of the products recognized actin [Median SFU 0.5/1×10⁵ cells (range 0-1)]. Relative or comparative immunodominance of these peptides may be determined from the values in Table 4.

### Example 4. HPIV3 Epitopes Demonstrate Cross-Reactivity with HPIV1

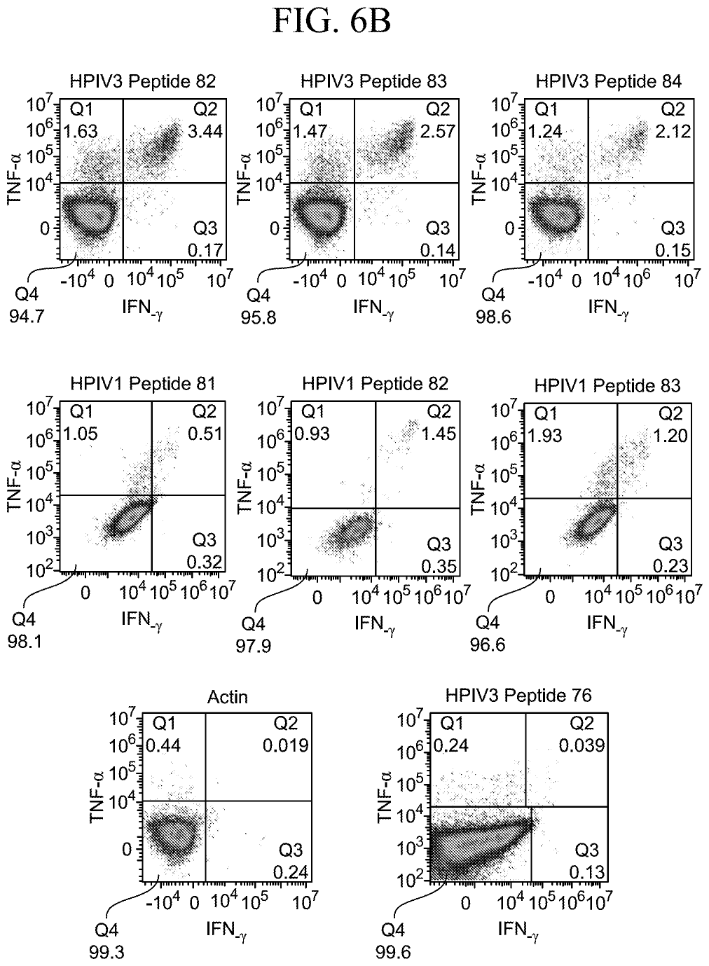

Given the homology between HPIV3 and related Respirovirus Human parainfluenza virus 1 the inventors sought to determine if immunodominant epitopes identified within HPIV3 Matrix protein were cross reactive with corresponding Human Parainfluenza Virus 1 epitopes. They initially tested overall specificity of HPIV3 specific T cell products for HPIV1 Matrix pepmix compared to HPIV3 Matrix pepmix using anti-IFN-g ELISpot. The mean response from all 10 products elicited by HPIV3 pepmix was 319.8 SFU/1×10⁵ cells, while the mean response elicited by HPIV1 pepmix was 138.9 SFU/1×10⁵ cells. Median actin, used as negative control, was 0.5 SFC/1×10⁵ cells (range 0-1). This resulted in a cross-reactivity index (HPIV1 response/HPIV3 response) of 0.4. We The cross-reactivity of HPIV3 specific T-cells against HPIV1 peptides using anti-IFN-g ELISpot. Cross-reactivity indices (HPIV1 mean SFU/HPIV3 mean SFU) ranged from 0.03 to 4.5 (Table 2). There was a high level of cross-reactivity between HPIV3 Peptides 83-84 and HPIV1 Peptides 82-83, with a cross-reactivity index of 1 and 1.07, respectively. HPIV1 Peptides 37 and 38 elicited a stronger response from HPIV3 specific T-cells than the corresponding HPIV3 epitopes, with cross-reactivity indices of 2.71 and 4.5, respectively. Of the 12 identified epitopes, 7 to 13 out of 15 amino acid sequences were conserved between strains (FIG. 6A).

Intracellular cytokine staining flow cytometry showed a predominant CD4+ response to Matrix peptides 38-39, 50, 59-60, 77-78, and 82-85 (FIG. 6B). Responding CD4+ cells demonstrated polyfunctional cytokine production, with a mean of 0.77% of cells producing IFN-g and TNF-α (range 0.03%-3.44%) compared to 0.09/o of CD8+ cells (range 0%-0.39%). Peptide 76 elicited a weak CD8+ response (mean 0.37%±0.015%)

The T-cells specific to the HPIV3 matrix epitopes disclosed in Example 1 were examined for their cross-reactivity with their corresponding HPIV1 matrix epitopes. The results are provided in Table 2, which clearly show cross reactivity between HPIV3 and HPIV1.

TABLE 2
Cross-reactivity of HPIV3-specific T-cells with HPIV1 matrix epitopes

| HPIV3 | | Amino | Corresponding | | Amino | Shared | Cross |
|---|---|---|---|---|---|---|---|
| matrix | | acid | HPIV1 | | acid | amino | reactivity |
| epitope | SEQ ID | sequence | matrix epitope | SEQ ID | sequence | acids | index |
| Peptide 38 | 7 | [symbol] | Peptide [symbol] | 20 | [symbol] | 12 | [symbol] |
| Peptide 39 | 8 | [symbol] | Peptide [symbol] | 21 | [symbol] | [symbol] | 4.5 |
| Peptide 50 | 9 | [symbol] | Peptide [symbol] | 22 | [symbol] | 7 | 0.03 |
| Peptide [symbol] | 10 | [symbol] | Peptide [symbol] | 23 | [symbol] | 10 | 0.3 |
| Peptide [symbol] | 11 | [symbol] | Peptide [symbol] | 24 | [symbol] | 10 | [symbol] |
| Peptide [symbol] | 13 | [symbol] | Peptide [symbol] | 25 | [symbol] | [symbol] | [symbol] |
| Peptide [symbol] | 14 | [symbol] | Peptide [symbol] | 26 | [symbol] | 12 | [symbol] |
| Peptide [symbol] | 15 | [symbol] | Peptide [symbol] | 27 | [symbol] | 12 | [symbol] |
| Peptide [symbol] | 16 | [symbol] | Peptide [symbol] | 28 | [symbol] | 11 | [symbol] |
| Peptide [symbol] | 17 | [symbol] | Peptide [symbol] | 29 | [symbol] | 10 | 1 |
| Peptide [symbol] | 18 | [symbol] | Peptide [symbol] | 30 | [symbol] | 10 | 1.07 |
| Peptide [symbol] | 19 | [symbol] | Peptide [symbol] | 31 | [symbol] | 9 | [symbol] |

[symbol]

[symbol]

indicates data missing or illegible when filed

Cross-reactivity between HPIV3 and HPIV1 is also depicted by FIGS. 6A and 6B as described above. In FIG. 6A, virus-specific T-cells (VSTs) were stimulated with individual HPIV3 peptides and corresponding HPIV1 peptides. Response was measured as spots per well (SFU/1×10⁵ cells) by anti-IFN-g ELISpot assay, and mean responses were calculated among responding products. Black bars represent HPIV3 peptides and gray bars represent HPIV1 peptides.

In FIG. 69, CD4+ T Cell Responses against HPIV3 and HPIV1 peptides were measured. Intracellular flow cytometry was performed on VSTs following stimulation with Actin, SEB, HPIV3 or HPIV1 peptides and anti-CD28/CD49. After 2 h stimulation with peptides, Brefeldin A was added for an additional 4 h following which cells were labeled with dead cell exclusion dye, Fe Receptor block and antibodies against CD3, CD4, CD8, TNF-a, IFN-g, and CD95. Following fixation and permeabilization cells were labeled intracellularly with antibodies targeting TNF-a, IFN-g, Percentage of CD4+ and CD8+ T-cells producing both IFNg+ and/or TNFα+ were measured.

### Example 5. HLA Class II Alleles that Identify Peptide Sequences of Novel CD4+ T-Cell Epitopes

The peptide epitopes identified in HPIV3 matrix protein were evaluated by in silico testing (Immune Epitope Database, IEDB.org, incorporated by reference). The in silico testing predicted that the peptides would have MHC class II restricted responses primarily mediated through HLA-DRB1 alleles (Table 3) and when stimulated with Matrix peptides, all products demonstrated a CD4+ response. These data can be used to select patients most responsive to T cell therapy using particular HPIV3 matrix protein epitopes or patients for selection of T cells suitable for ex vivo induction of HPIV3-specific T cells using these peptides epitopes.

TABLE 3
HLA Class II alleles that identify peptide sequences of CD4+ T-cell epitopes

| | | | | | | | IEDP MHC-II |
|---|---|---|---|---|---|---|---|
| Peptide # | Peptide sequence | SEQ ID NO. 3 [symbol] | Product # | HLA- [symbol] | HLA- [symbol] | HLA- [symbol] | Ending Prediction |
| 38 | [symbol] | [symbol] | 4 | [symbol] | [symbol] | [symbol] | [symbol] |
| 39 | [symbol] | [symbol] | 4 | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | | | | [symbol] | [symbol] | [symbol] | |
| [symbol] | [symbol] | [symbol] | [symbol] | [symbol] | [symbol] | [symbol] | [symbol] |
| | | | | [symbol] | [symbol] | [symbol] | |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | | | | [symbol] | [symbol] | [symbol] | |
| [symbol] | [symbol] | [symbol] | [symbol] | [symbol] | [symbol] | [symbol] | [symbol] |
| 82 | [symbol] | [symbol] | [symbol] | [symbol] | [symbol] | [symbol] | [symbol] |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| 83 | [symbol] | [symbol] | [symbol] | [symbol] | [symbol] | [symbol] | [symbol] |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| 84 | [symbol] | [symbol] | [symbol] | [symbol] | [symbol] | [symbol] | [symbol] |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| | | | [symbol] | [symbol] | [symbol] | [symbol] | |
| 85 | [symbol] | [symbol] | [symbol] | [symbol] | [symbol] | [symbol] | [symbol] |
| | | | [symbol] | [symbol] | [symbol] | [symbol] | |

[symbol]

indicates data missing or illegible when filed

[symbol]

### Example 6. Immunodominance of HPIV3 Epitopes

The selection of immunodominant T cell epitopes of HPIV3 is useful for selecting HPIV3-specific T cell populations for treatment of HPIV3 disease or for formulation of a potent vaccine against HPIV3. The identification of immunodominant HPIV3 epitopes is essential for selection of partially-HLA matched VSTs, and development of third-party HPIV3 VST therapy. To evaluate immunodominance the responsiveness (number of responders, mean SFU) of T cells often subjects (10 products) were evaluated against the 12 T cell epitopes identified by the inventors. Seven of the 12 peptides were recognized by multiple donors. A highly dominant epitope was identified within the group, GEFRYYPNIIAKGVG (SEQ ID NO: 18), which elicited anti-viral activity from all 10 products as shown by Table 4. These data permit one to select peptides likely to induce T cell responses against HPIV3 in a broader class of patients and induce more potent responses. They also permit the formulation of 2, 3, 4 or more HPIV3-specific T cells for broader and more potent protection against HPIV3 infection, for example, by including the more immunodominant T cells such as Peptides 84, 83, or 82 in a formulation or by including these more immunodominant T cell peptides in a vaccine or immunogenic composition for inducing HPIV3-specific T cells in vivo or ex vivo.

TABLE 4
Hierarchy of immunodominance of HPIV3 Matrix Protein Epitopes

| Peptide # | SEQ ID NO. 3 residues | Epitope | [symbol] | [symbol] | [symbol] |
|---|---|---|---|---|---|
| [symbol] | [symbol] | [symbol] | 10 | [symbol] | [symbol] |
| [symbol] | [symbol] | [symbol] | 9 | [symbol] | [symbol] |
| [symbol] | [symbol] | [symbol] | 7 | [symbol] | [symbol] |
| [symbol] | [symbol] | [symbol] | 2 | [symbol] | [symbol] |
| [symbol] | [symbol] | [symbol] | 2 | [symbol] | [symbol] |
| [symbol] | [symbol] | [symbol] | 2 | [symbol] | [symbol] |
| [symbol] | [symbol] | [symbol] | 2 | [symbol] | [symbol] |
| [symbol] | [symbol] | [symbol] | 1 | [symbol] | [symbol] |
| [symbol] | [symbol] | [symbol] | 1 | [symbol] | [symbol] |
| [symbol] | [symbol] | [symbol] | 1 | [symbol] | [symbol] |
| [symbol] | [symbol] | [symbol] | 1 | [symbol] | [symbol] |
| [symbol] | [symbol] | [symbol] | 1 | [symbol] | [symbol] |

[symbol]

indicates data missing or illegible when filed

[symbol]

### Example 7. HPIV3 Epitope Mapping

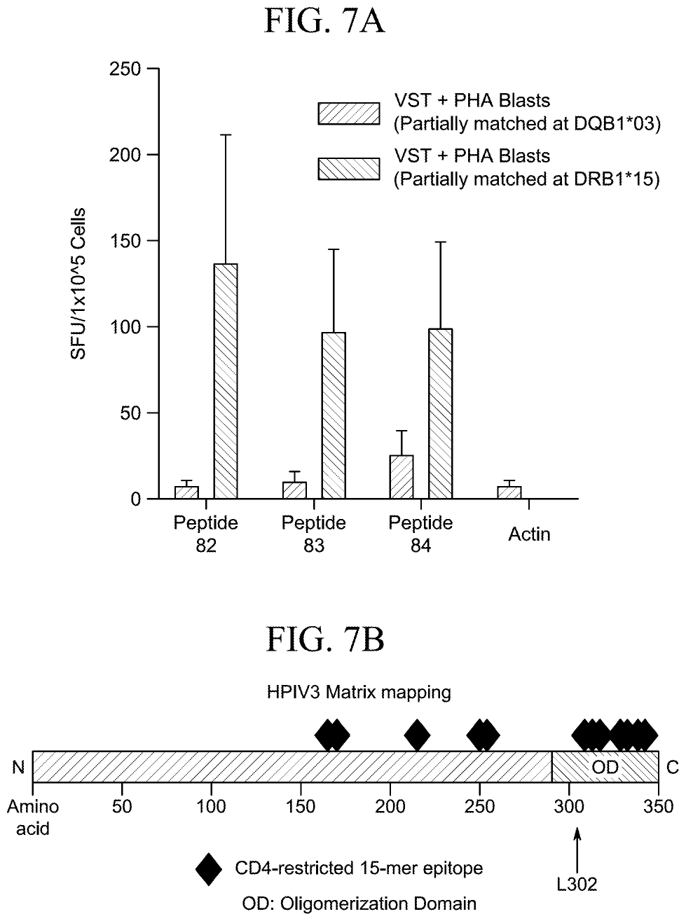

The 12 epitopes identified and characterized by the inventors were mapped on the HPIV3 matrix protein. FIG. 7A shows the response (SFU) of VST+PHA blasts partially matched at DQB1′*03 or DRB1*15 to immunodominant Peptides 82, 83 and 84. The location of the 12 peptide epitopes in HPIV3 matrix protein is shown by FIG. 7B. The oligomerization domain at the C-terminus contains 7 out of 12 epitopes, including the three most immunodominant peptides, 82-84. The black arrow denotes the L302 residue, which is a target of ubiquitination during virion production. These data permit further targeting of T cell responses to HPIV3 matrix protein at the C terminal and especially in the oligomerization domain.

The findings disclosed herein demonstrate the importance of CD4+ T cells in their role of respiratory virus clearance. Epitopes induced polyfunctional responses based on TNF-α and IFN-g production. In silico predictions suggested that the most immunodominant peptide, GEFRYYPNIIAKGVG (SEQ. ID NO: 18), would elicit a T cell response restricted through HLADRB1*15:01. Only 2 out of 10 products were matched at that allele, but restriction analysis showed that the VSTs responded to PHA blast targets matched at HLA-DRB1*15:01. The inventors also showed that IFN-gamma production was decreased by 32%-80% in the presence of HLA-DR blocking antibodies. Similar findings were noted in CD4+ responses against MHC Class H-restricted adenovirus Hexon epitopes, where the T-cell response of multiple T cell products with differing HLA-alleles was blocked by an HLA-DR antibody. Our findings suggest that while there is some degree of HLA-DR restriction, T cell activity against GEFRYYPNIIAKGVG (SEQ. ID NO: 18) is likely not limited to a single Class II allele. Although the T cell products were manufactured from a diverse group of donors, a limitation to this study is that we cannot evaluate for all possible HLA restricted responses. However, we and others have shown that even knowing a relatively restricted number of class I and class II restricted virus-specific responses provides important clinical information when evaluating virus-specific T cell products in a third party “off the shelf” setting.

Previous studies have shown that cytotoxic T-cells stimulated by HPIV1-infected cells also recognize HPIV3 proteins. Additional studies showed that while antibody cross-reactivity is minimal within the paramyxovirus family, T cell cross-reactivity is common between HPIV3 and other related viruses such as measles, mumps, and RSV. Since HPIV3 and HPIV1 both occur in an immunocompromised population, peptide sequences from HPIV1 corresponding to the amino acid sequences from the identified HPIV3 epitopes were tested and confirmed cross-reactivity in of 12 epitopes. The highest degree of cross-reactivity was between HPIV3 epitopes 83 and 84 and the corresponding HPIV1 epitopes (82 and 83, respectively), which were the epitopes recognized most consistently by the T cell products.

As shown herein, the inventors have characterized multiple novel immunodominant epitopes within HPIV3 Matrix protein. These HPIV3 epitopes show a high degree of cross-reactivity with corresponding HPIV1 epitopes. The immunodominance and cross-reactivity with HPIV1 of these responses are consistent with epitopes serving important roles in the adaptive immune response to human parainfluenza viruses. This disclosure permits the formulation of specific third party HPIV3 and HPIV1-VST products to rapidly treat immunocompromised individuals infected with these lethal respiratory viruses.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A method for preventing or treating an infection with human parainfluenza virus 3 (“HPIV3”), comprising administering to a subject in need thereof an isolated subpopulation of T cells recognizing one or more peptide epitopes of HPIV3.

2. The method of claim 1, wherein said subject is infected with HPIV3.

3. The method of claim 1, wherein said subject is asymptomatic or is at risk of exposure to HPIV3.

4. The method of claim 1, wherein the subpopulation of T cells was ex vivo primed to an HPIV3 peptide epitope and/or expanded prior to said administering.

5. The method of claim 1, wherein the T cells are autologous to the subject.

6. The method of claim 1, wherein said T cells are allogeneic to the subject and comprise at least one HLA class I or class II allele in common with the subject.

7. The method of claim 1, wherein said T cells share with the subject at least one HLA class II allele selected from the group consisting of HLA-DR, HLA-DQ or HLA-DP.

8. The method of claim 1, wherein said T cells share with the subject at least one HLA class II allele selected from the group consisting of HLA-DRB1, HLA-DRB3 or HLA-DPB1.

9. The method of claim 1, wherein said T cells share at least one HLA class II allele selected from the group consisting of HLA-DRB3*02.02, HLA-DRB1*01.01, HLA-DRB1*01.01, HLA-DRB1*01.01, HLA-DRB1*01.01, HLA-DRB1*01.01, HLA-DRB1*01.01, HLA-DRB1*01.01, or HLA-DRB1*01.01.

10. The method of claim 1, wherein the one or more peptide epitopes of HPIV3 are contained within Peptide 84, Peptide 83, Peptide 82, Peptide 59, Peptide 85, Peptide 60, Peptide 76, Peptide 78, Peptide 38, Peptide 77, Peptide 39 or Peptide 50; or wherein the one or more peptide epitopes of HPIV3 comprise one or two insertions, substitutions or deletions to an amino acid sequence of Peptide 84, Peptide 83, Peptide 82, Peptide 59, Peptide 85, Peptide 60, Peptide 76, Peptide 78, Peptide 38, Peptide 77, Peptide 39 or Peptide 50.

11. The method of claim 1, wherein at least two isolated subpopulations T cells that recognize at least different peptide epitopes of HPIV3 are administered to the subject in need thereof, and wherein at least one of the peptide epitopes is contained in Peptide 84, 83 or 82.

12. The method of claim 1, wherein the one or more peptide epitopes of HPIV3 are contained within Peptide 84, Peptide 83, Peptide 82, Peptide 59, Peptide 85, Peptide 60, Peptide 76, Peptide 78, Peptide 38, Peptide 77, Peptide 39 or Peptide 50; or wherein the one or more peptide epitopes of HPIV3 comprise one or two insertions, substitutions or deletions to an amino acid sequence of Peptide 84, Peptide 83, Peptide 82, Peptide 59, Peptide 85, Peptide 60, Peptide 76, Peptide 78, Peptide 38, Peptide 77, Peptide 39 or Peptide 50; and wherein the T cells are optionally contacted with said one or more peptide epitopes ex vivo, and optionally expanded, prior to said administering.

13. The method of claim 1, wherein the T cells comprise one or a plurality of banked T-cell subpopulations.

14. The method of claim 1, further comprising determining HLA subtype(s) of the subject and selecting one or a plurality of banked T-cell subpopulations having activity against an HPIV3 peptide epitope restricted by said HLA subtype(s), and administering said selected T cell subpopulations to the subject.

15. A composition comprising at least one isolated subpopulation of T cells recognizing one or more restricted peptide epitopes of HPIV3, wherein the at least one isolated subpopulation of T cells was ex vivo primed to an HPIV3 peptide epitope.

16. (canceled)

17. (canceled)

18. The composition of claim 15, wherein the subpopulation(s) of T cells recognizes one or more peptide epitopes contained in Peptide 84, Peptide 83, Peptide 82, Peptide 59, Peptide 85, Peptide 60, Peptide 76, Peptide 78, Peptide 38, Peptide 77, Peptide 39 or Peptide 50.

19. (canceled)

20. (canceled)

21. (canceled)

22. (canceled)

23. (canceled)

24. A method for priming or expanding in vivo at least one subpopulation of T cells recognizing HPIV3 epitopes comprising contacting T cells gf a subject at least one of Peptide 84, Peptide 83, Peptide 82, Peptide 59, Peptide 85, Peptide 60, Peptide 76, Peptide 78, Peptide 38, Peptide 77, Peptide 39 or Peptide 50.

25. (canceled)

26. (canceled)

27. (canceled)

28. The method of claim 24, further comprising isolating the at least one subpopulation of T cells that recognize HLA restricted peptide epitopes of HPIV3,
wherein said at least one of Peptide 84, Peptide 83, Peptide 82, Peptide 59, Peptide 85, Peptide 60, Peptide 76, Peptide 78, Peptide 38, Peptide 77, Peptide 39 or Peptide 50 is part of an overlapping peptide library, and
wherein said contacting occurs in the presence of dendritic or other antigen processing cells that present the at least one of Peptide 84, Peptide 83, Peptide 82, Peptide 59, Peptide 85, Peptide 60, Peptide 76, Peptide 78, Peptide 38, Peptide 77, Peptide 39 or Peptide 50.

29. The method of claim 28, further comprising contacting said T cells in combination with the overlapping peptide library with IL-4 and IL-7.

30. The method of claim 28, wherein said overlapping peptide library is for HPIV3 matrix protein and the isolated T cells recognize a peptide epitope contained within Peptide 84, Peptide 83, Peptide 82, Peptide 59, Peptide 85, Peptide 60, Peptide 76, Peptide 78, Peptide 38, Peptide 77, Peptide 39 or Peptide 50.


SEQUENCE LISTING (31 sequences)

SEQ ID NO: 1 - protein, 572 aa, Parainfluenza virus type 3
  MISC_FEATURE (1)..(572): HN, strain Wash/47885/57
MEYWKHTNHGKDAGNELETSMATHGNKITNKITYILWTIILVLLSIVFIIVLINSIKSEKAHESLLQDVNNEFMEVTEKIQMASDNINDLIQSGVNTRLLTIQSHVQNYIPISLTQQMSDLRKFISEITIRNDNQEVPPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNHDGSISTTRFKNNNISFDQPYAALYPSVGPGIYYKGKIIFLGYGGLEHPINENAICNTTGCPGKTQRDCNQASHSPWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTSTERVNELAIRNKTLSAGYTTTSCITHYNKGYCFHIVEINHKSLDTFQPMLFKTEIPKSCS

SEQ ID NO: 2 - protein, 539 aa, Parainfluenza virus type 3
  MISC_FEATURE (1)..(539): Fus, strain Wash/47885/57
MPTSILLIITTMIMASFCQIDITKLQHVGVLVNSPKGMKISQNFETRYLILSLIPKIEDSNSCGDQQIKQYKRLLDRLIIPLYDGLRLQKDVIVSNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYRVDSISYNIQNREWYIPLPSHIMTKGAFLGGADVKECIEAFSSYICPSDPGFVLNHEMESCLSGNISQCPRTVVKSDIVPRYAFVNGGVVANCITTTCTCNGIGNRINQPPDQGVKIITHKECNTIGINGMLFNTNKEGTLAFYTPNDITLNNSVALDPIDISIELNKAKSDLEESKEWIRRSNQKLDSIGNWHQSSTTIIIVLIMIIILFIINVTIIIIAVKYYRIQKRNRVDQNDKPYVLTNK

SEQ ID NO: 3 - protein, 353 aa, Parainfluenza virus type 3
  MISC_FEATURE (1)..(353): Mat, strain Wash/47885/57
MSITNSAIYTFPESSFSENGHIEPLPLKVNEQRKAVPHIRVAKIGNPPKHGSRYLDVFLLGFFEMERIKDKYGSVNDLDSDPGYKVCGSGSLPIGLAKYTGNDQELLQAATKLDIEVRRTVKAKEMIVYTVQNIKPELYPWSSRLRKGMLFDANKVALAPQCLPLDRSIKFRVIFVNCTAIGSITLFKIPKSMASLSLPSTISINLQVHIKTGVQTDSKGIVQILDEKGEKSLNFMVHLGLIKRKVGRMYSVEYCKQKIEKMRLIFSLGLVGGISLHVNATGSISKTLASQLVFKREICYPLMDLNPHLNLVIWASSVEITRVDAIFQPSLPGEFRYYPNIIAKGVGKIKQWN

SEQ ID NO: 4 - protein, 515 aa, Parainfluenza virus type 3
  MISC_FEATURE (1)..(515): NP, strain Wash/47885/57
MLSLFDTFNARRQENITKSAGGAIIPGQKNTVSIFALGPTITDDNEKMTLALLFLSHSLDNEKQHAQRAGFLVSLLSMAYANPELYLTTNGSNADVKYVIYMIEKDLKRQKYGGFVVKTREMIYEKTTEWIFGSDLDYDQETMLQNGRNNSTIEDLVHTFGYPSCLGALIIQIWIVLVKAITSISGLRKGFFTRLEAFRQDGTVQAGLVLSGDTVDQIGSIMRSQQSLVTLMVETLITMNTSRNDLTTIEKNIQIVGNYIRDAGLASFFNTIRYGIETRMAALTLSTLRPDINRLKALMELYLSKGPRAPFICILRDPIHGEFAPGNYPAIWSYAMGVAVVQNRAMQQYVTGRSYLDIDMFQLGQAVARDAEAQMSSTLEDELGVTHEAKESLKRHIRNINSSETSFHKPTGGSAIEMAIDEEPEQFEHRADQEQDGEPQSSIIQYAWAEGNRSDDRTEQATESDNIKTEQQNIRDRLNKRLNDKKKQGSQPSTNPTNRTNQDEIDDLFNAFGSN

SEQ ID NO: 5 - protein, 604 aa, Parainfluenza virus type 3
  MISC_FEATURE (1)..(604): P, strain Wash/47885/57
MESDAKNYQIMDSWEEEPRDKSTNISSALNIIEFILSTDPQEDLSENDTINTRTQQLSATICQPEIKPTETSEKVSGSTDKNRQSGSSHECTTEAKDRNIDQETVQGGSGRRSSSDSRAETVVSGGISGSITDSKNGTQNTENIDLNEIRKMDKDSIERKMRQSADVPSEISGSDVIFTTEQSRNSDHGRSLEPISTPDTRSMSVVTAATPDDEEEILMKNSRMKKSSSTHQEDDKRIKKGGGGKGKDWFKKSRDTDNQTSTSDHKPTSKGQKKISKTTTTNTDTKGQTETQTESSETQSPSWNPIIDNNTDRTEQTSTTPPTTTPRSTRTKESIRTNSESKPKTQKTIGKERKDTEESNRFTERAITLLQNLGVIQSTSKLDLYQDKRVVCVANVLNNVDTASKIDFLAGLVIGVSMDNDTKLIQIQNEMLNLKADLKRMDESHRRLIENQREQLSLITSLISNLKIMTERGGKKDQNESNERVSMIKTKLKEEKIKKTRFDPLMEAQGIDKNIPDLYRHAGNTLENDVQVKSEILSSYNESNATRLIPRKVSSTMRSLVAVINNSNLPQSTKQSYINELKHCKSDEEVSELMDMFNEDVNNC

SEQ ID NO: 6 - protein, 2233 aa, Parainfluenza virus type 3
  MISC_FEATURE (1)..(2233): L, strain Wash/47885/57
MDTESNNGTVSDILYPECHLNSPIVKGKIAQLHTIMSLPQPYDMDDDSILVITRQKIKLNKLDKRQRSIRRLKLILTEKVNDLGKYTFIRYPEMSKEMFKLHIPGINSKVTELLLKADRTYSQMTDGLRDLWINVLSKLASKNDGSNYDLNEEINNISKVHTTYKSDKWYNPFKTWFTIKYDMRRLQKARNEVTFNMGKDYNLLEDQKNFLLIHPELVLILDKQNYNGYLITPELVLPYCDVVEGRWNISACAKLDPKLQSMYQKGNNLWEVIDKLFPIMGEKTFDVISLLEPLALSLIQTHDPVKQLRGAFLNHVLSEMELIFESRESIKEFLSVDYIDKILDIFNKSTIDEIAEIFSFFRTFGHPPLEASIAAEKVRKYMYIGKQLKFDTINKCHAIFCTIIINGYRERHGGQWPPVTLPDHAHEFIINAYGSNSAISYENAVDYYQSFIGIKFNKFIEPQLDEDLTIYMKDKALSPKKSNWDTVSPASNLLYRTNASNESRRLVEKFIADSKFDPNQILDYVESGDWLDDPEFNISYSLKEKEIKQEGRLFAKMTYKMRATQVLSETLLANNIGKFFQENGMVKGEIELLKRLTTISISGVPRYNEVYNNSKSHTDDLKTYNKISNLNLSSNQKSKKFEFKSTDIYNDGYETVSCFLTTDLKKYCLNWRYESTALFGETCNQIFGLNKLFNWLHPRLEGSTIYVGDPYCPPSDKEHISLEDHPDSGFYVHNPRGGIEGFCQKLWTLISISAIHLAAVRIGVRVTAMVQGDNQAIAVTTRVPNNYDYRVKKEIVYKDVVRFFDSLREVMDDLGHELKLNETIISSKMFIYSKRIYYDGRILPQALKALSRCVFWSETVIDETRSASSNLATSFAKAIENGYSPVLGYACSIFKNIQQLYIALGMNINPTITQNIKDLYFRNPNWMQYASLIPASVGGFNYMAMSRCFVRNIGDPSVAALADIKRFIKANLLDRSVLYRIMNQEPGESSFLDWASDPYSCNLPQSQNITTMIKNITARNVLQDSPNPLLSGLFTNTMIEEDEELAEFLMDRKVILPRVAHDILDNSLTGIRNAIAGMLDTTKSLIRVGINRGGLTYSLLRKISNYDLVQYETLSRTLRLIVSDKIRYEDMCSVDLAIALRQKMWIHLSGGRMISGLETPDPLELLSGVIITGSEHCKICYSSDGTNPYTWMYLPGNIKIGSAETGISSLRVPYFGSVTDERSEAQLGYIKNLSKPAKAAIRIAMIYTWAFGNDEISWMEASQIAQTRANFTLDSLKILTPVATSTNLSHRLKDTATQMKFSSTSLIRVSRFITMSNDNMSIKEANETKDTNLIYQQIMLTGLSVFEYLFRLEETTGHNPIVMHLHIEDECCIKESFNDEHINPESTLELIRYPESNEFIYDKDPLKDVDLSKLMVIKDHSYTIDMNYWDDTDIIHAISICTAITIADTMSQLDRDNLKEIIVIANDDDINSLITEFLTLDILVFLKTFGGLLVNQFAYTLYSLKTEGRDLIWDYIMRTLRDTSHSILKVLSNALSHPKVFKRFWDCGVLNPIYGPNTASQDQIKLALSICEYSLDLFMREWLNGVSLEIYICDSDMEVANDRKQAFISRHLSFVCCLAEIASFGPNLLNLTYLERLDLLKQYLELNIKDDPTLKYVQISGLLIKSFPSTVTYVRKTAIKYLRIRGISPPEVIDDWDPIEDENMLDNIVKTINDNCNKDNKGNKINNFWGLALKNYQVLKIRSITSDSDNNDRSDASTGGLTLPQGGNYLSHQLRLFGINSTSCLKALELSQILMKEVNKDQDRLFLGEGAGAMLACYDATLGPAVNYYNSGLNITDVIGQRELKIFPSEVSLVGKKLGNVTQILNRVKVLFNGNPNSTWIGNMECETLIWSELNDKSIGLVHCDMEGAIGKSEETVLHEHYSVIRITYLIGDDDVVLISKIIPTITPNWSRILYLYKLYWKDVSIISLKTSNPASTELYLISKDAYCTIMEPSEVVLSKLKRLSLLEENNLLKWIILSKKKNNEWLHHEIKEGERDYGVMRPYHMALQIFGFQINLNHLAKEFLSTPDLTNINNIIQSFQRTIKDVLFEWINITHDGKRHKLGGRYNIFPLKNKGKLRLLSRRLVLSWISLSLSTRLLTGRFPDEKFEHRAQTGYVSLPDTDLESLKLLSKNTIKNYRECIGSISYWFLTKEVKILMKLIGGAKLLGIPRQYKEPEEQLLEDYNQHDEFDID

SEQ ID NO: 7 - protein, 15 aa, Artificial Sequence
  Peptide 38
MLFDANKVALAPQCL

SEQ ID NO: 8 - protein, 15 aa, Artificial Sequence
  Peptide 39
ANKVALAPQCLPLDR

SEQ ID NO: 9 - protein, 15 aa, Artificial Sequence
  Peptide 50
SLPSTISINLQVHIK

SEQ ID NO: 10 - protein, 15 aa, Artificial Sequence
  Peptide 59
LNFMVHLGLIKRKVG

SEQ ID NO: 11 - protein, 15 aa, Artificial Sequence
  Peptide 60
VHLGLIKRKVGRMYS

SEQ ID NO: 12 - protein, 15 aa, Artificial Sequence
  Peptide 75
EICYPLMDLNPHLNL

SEQ ID NO: 13 - protein, 15 aa, Artificial Sequence
  Peptide 76
PLMDLNPHLNLVIWA

SEQ ID NO: 14 - protein, 15 aa, Artificial Sequence
  Peptide 77
LNPHLNLVIWASSVE

SEQ ID NO: 15 - protein, 15 aa, Artificial Sequence
  Peptide 78
LNLVIWASSVEITRV

SEQ ID NO: 16 - protein, 15 aa, Artificial Sequence
  Peptide 82
AIFQPSLPGEFRYYP

SEQ ID NO: 17 - protein, 15 aa, Artificial Sequence
  Peptide 83
PSLPGEFRYYPNIIA

SEQ ID NO: 18 - protein, 15 aa, Artificial Sequence
  Peptide 84
GEFRYYPNIIAKGVG

SEQ ID NO: 19 - protein, 15 aa, Artificial Sequence
  Peptide 85
YYPNIIAKGVGKIKQ

SEQ ID NO: 20 - protein, 15 aa, Artificial Sequence
  HPIV1 Peptide 37
MIYNANKVALAPQCL

SEQ ID NO: 21 - protein, 15 aa, Artificial Sequence
  HPIV1 Peptide 38
ANKVALAPQCLPVDK

SEQ ID NO: 22 - protein, 15 aa, Artificial Sequence
  HPIV1 Peptide 49
ALPNSISVNLLVTLR

SEQ ID NO: 23 - protein, 15 aa, Artificial Sequence
  HPIV1 Peptide 58
LMFMVHLGIIRRKVG

SEQ ID NO: 24 - protein, 15 aa, Artificial Sequence
  HPIV1 Peptide 59
VHLGIIRRKVGKIYS

SEQ ID NO: 25 - protein, 15 aa, Artificial Sequence
  HPIV1 Peptide 75
PLMDVNPHMNLVIWA

SEQ ID NO: 26 - protein, 15 aa, Artificial Sequence
  HPIV1 Peptide 76
VNPHMNLVIWAASVE

SEQ ID NO: 27 - protein, 15 aa, Artificial Sequence
  HPIV1 Peptide 77
MNLVIWAASVEITSV

SEQ ID NO: 28 - protein, 15 aa, Artificial Sequence
  HPIV1 Peptide 81
AVFQPAIPKEFRYYP

SEQ ID NO: 29 - protein, 15 aa, Artificial Sequence
  HPIV1 Peptide 82
PAIPKEFRYYPNVVA

SEQ ID NO: 30 - protein, 15 aa, Artificial Sequence
  HPIV1 Peptide 83
KEFRYYPNVVAKSIG

SEQ ID NO: 31 - protein, 15 aa, Artificial Sequence
  HPIV1 Peptide 84
YYPNVVAKSIGKIRR